(19) United States
(12) Patent Application Publication
(10) Pub. No.: US 2024/0269282 A1
(43) Pub. Date: Aug. 15, 2024

(54) Methods for the Treatment of Cancer, Inflammatory Diseases and Autoimmune Diseases

(71) Applicants: Institut Curie, Paris (FR); INSERM (Institut National de la Santé et de la Recherche Médicale), Paris (FR)

(72) Inventors: Clotilde Thery, Paris (FR); Mercedes Tkach, Bourg-la-Reine (FR)

(73) Assignees: Institut Curie, Paris (FR); INSERM (Institut National de la Santé et de la Recherche Médicale), Paris (FR)

(21) Appl. No.: 18/290,153

(22) PCT Filed: May 10, 2022
(86) PCT No.: PCT/EP2022/062607

§ 371 Date: Nov. 10, 2023

(30) Foreign Application Priority Data
May 10, 2021 (EP) 21305600.5

(51) Int. Cl. A61K 39/00; A61K 9/50; A61P 35/00; C07K 14/725; C07K 16/30; C12N 5/0786

(52) U.S. Cl. CPC A61K 39/4614; A61K 9/5068; A61K 39/4631; A61K 39/4644; A61P 35/00; C07K 14/7051; C07K 16/30; C12N 5/0645; C12N 2510/00

(57) ABSTRACT

The invention relates to methods and pharmaceutical compositions for the treatment and diagnosis of cancer. The invention also relates to methods and pharmaceutical compositions for the treatment of inflammatory diseases and autoimmune diseases. The inventors investigate the role and specific contribution of extracellular vesicles (EVs) in cancer environment. The inventors demonstrate that CSF1-associated EVs induce macrophage signature associated with T cell infiltration and extended patient survival. The inventors demonstrate that via specific extracellular vesicles, these tumors promote pro-inflammatory macrophages correlated with better clinical outcome and a better prognosis in TNBC patients. In the present invention, the inventors provide in vitro evidences towards a direct role of CSF1-associated EVs as tools, alone or with other immuno-therapies, to promote anti-tumor immune responses. Thus, the present invention relates to CSF1-associated EVs, their use in the treatment and diagnosis of cancer, and their targeting in the treatment of inflammatory diseases and autoimmune diseases.

### FIELD OF THE INVENTION

The invention relates to the field of immunotherapy. The invention relates to methods and pharmaceutical compositions for the treatment and diagnosis of cancer, inflammatory diseases or autoimmune diseases.

### BACKGROUND OF THE INVENTION

Tumors are infiltrated by different populations of immune cells, macrophages in particular being major components of the tumor microenvironment. Tumor-associated macrophages (TAMs) favor tumor progression, promoting cancer cell invasion and metastasis in mouse models (Cassetta and Pollard, 2018). Recent single-cell analyses of human cancers revealed the heterogeneity of macrophage populations, thus challenging our understanding of TAM biology (Azizi et al., 2018; Chevrier et al., 2017; Lavin et al., 2017). TAMs derive from circulating monocytes that are recruited into the tumor via the CCL2-CCR2 chemokine signaling pathway (Franklin et al., 2014; Qian et al., 2011). The fate of monocytes is not predetermined and largely depend on the microenvironmental cues they encounter (Goudot et al., 2017). Specifically, the identity of tumor derived factors that contribute to TAM heterogeneity and the mechanisms underlying intratumoral monocyte differentiation remain unclear.

Among the signals that can impact TAM differentiation and activation, cytokines and chemokines are well-characterized factors (Cassetta et al., 2019); extracellular vesicles (EVs), however, represent novel candidates. EVs are complex vehicles of intercellular communication and were suggested to have an impact on macrophage activation (Chow et al., 2014; Haderk et al., 2017; Wu et al., 2016; Ying et al., 2016). EVs, such as exosomes, ectosomes, microvesicles, oncosomes, are membrane-enclosed structures that contain proteins and nucleic acids, and can be released into the extracellular environment by all cell types, including cancer cells (Tkach and Théry, 2016; van Niel et al., 2018). Once released, EVs can interact with recipient cells and modulate their function (Cocozza et al., 2020). Particularly, EVs released by cancer cells play an important role in shaping the tumor immune microenvironment. EVs were shown to modulate lymphocytes and myeloid cell functions in cancer by triggering either pro-tumor or anti-tumor immune responses (Kugeratski and Kalluri, 2021; Robbins and Morelli, 2014), which may depend on numerous factors, such as the cancer type, stage, or EV subtype analyzed (Tkach et al., 2018).

The inventors investigate the role and specific contribution of tumor-derived EVs, as compared to the tumor derived soluble factors, in driving TAM heterogeneity. For this, the inventors focus on human breast cancer since these tumors are highly infiltrated with macrophages (Cassetta and Pollard, 2018). The inventors confirm a striking functional difference of EVs and soluble factors in tuning TAM profile, which is attributed to the combination of survival and activation signals carried by EVs. Furthermore, the inventors unravel an unexpected ability of TNBC-EVs to promote an inflammatory tumor microenvironment associated with a better clinical outcome.

The inventors demonstrate that CSF1-associated EV promote a tumor immune microenvironment associated with a favourable prognosis in TNBC patients. There is no disclosure in the art of the role of CSF1-associated EV and their use in the treatment and diagnosis of cancer. There is also no disclosure in the art of the specific targeting of CSF1-associated EV in the treatment of inflammatory diseases and autoimmune diseases.

### SUMMARY OF THE INVENTION

The invention relates to methods and pharmaceutical compositions for the treatment and diagnosis of cancer. The invention also relates to methods and pharmaceutical compositions for the treatment of inflammatory diseases and auto-immune diseases. In particular, the invention is defined by the claims.

### SHORT DESCRIPTION OF THE DRAWINGS

These and further aspects of the invention will be explained in greater detail by way of examples and with reference to the accompanying drawings in which:

FIG. 1. Efficient separation of MDA-MB-231-derived secretome into EVs and soluble factors by ultrafiltration and SEC.

(A) Scheme of collection of the EV-enriched and EV-poor fractions. Conditioned medium from MDA-MB-231 cells cultured ON in serum free medium was concentrated by ultrafiltration with 100 KDa cut-off filters. 0.5 ml of the concentrated conditioned medium (CCM) was subjected to Size Exclusion Chromatography and 0.5 ml individual or pooled fractions were collected and concentrated using 10 KDa cut-off filters. (B) Vesicle count and protein quantification on individual fractions is shown. Representative of two independent experiments. (C) WB of EV-associated proteins done on individual fractions and transmission electron microscopy of the pooled EV-rich (EV-R) and EV-poor (EV-P) fractions. Scale bar=100 nm. Representative of three independent experiments. (D) Quantification of IL-6 and IL-8 present in pooled SEC fractions from MDA-MB-231 cells. Representative of two independent experiments. (E) MACSPlex Exosome on EV-containing fractions (F7-10) developed using a mix of antibodies against TSPs (CD9, CD63 and CD81). Results for individual EV isolations are shown (n=7).

FIG. 2. EVs and soluble factors from MDA-MB-231 cells promote the differentiation of monocytes towards macrophages.

(A) Equal amount of proteins (4 μg) from pooled EV-R (F7-10) or EV-poor (F15-22) fractions were incubated for 5 days with freshly isolated CD14+ monocytes from healthy donors in the absence of any other stimuli. As control, CD14+ cells were also incubated with 100 ng/ml of rCSF-1 or rGM-CSF. On day 5, live cells were counted on each well by flow cytometry. (B) Mo-derived cells morphology was analyzed by cytospin at the end of the culture (Day 5). Bars, 30 μm. Representative of two independent experiments. (C) Analysis of macrophage marker expression by flow cytometry. Representative of two (for CD68, CD88 and CD1a expression) and of three to ten independent donors for the other markers (isotype control on CSF-1-treated cells is shown in grey). (D) CD206 vs CD163 density plot of cells at day 5. Representative of four independent experiments. (E) Quantification of CD206+CD163+ live macrophages on day 5 of culture of monocytes with increasing doses of each pooled fraction (0.5, 1 and 2 μg of proteins). (F) Quantification of CD206+CD163+ live macrophages on day 5 of culture of monocytes with EV-R or EV-P fractions from equal numbers of control (CTRL gRNA) or Rab11a-KO (Rab11 gRNA) MDA-MB-231 cells. Each individual donor is shown (n=9). Comparison between groups was performed by two-tailed, Wilcoxon test. P values≤0.05 were considered significant and are indicated for each comparison. Each individual donor is shown. Results shown represent mean±SEM.

FIG. 3. EVs from TNBC MDA-MB-231 and BT-549 cells but not from luminal MCF-7 cells expose CSF-1 which is required for mo-macs induction

(A) Quantification of cytokines present in pooled SEC fractions from MDA-MB-231 cells measured by Flow Cytometry bead-based assays. Representative of two independent EV isolations. (B) Recovery of CSF-1 on MDA-MB-231 EVs by pull-down with anti-CD9, anti-CD81 and anti-CD63 (Pan-EV). (C) Presence of CSF-1 on lipid dye positive (membright 488) EVs measured by Imaging Flow cytometry (ImageStream-X™). (D) MACSPlexExo analysis of MDA-MB-231 EV-R fractions, developed using a fluorescently-coupled antibody against CSF-1. (E) Quantification of CD206+CD163+ live macrophages on day 5 of culture of monocytes with MDA-MB-231 EV-R in the presence of blocking antibodies against CSF-1 Receptor (CD115) or GM-CSF Receptor (CD116) molecules. Each individual donor is shown (n=6). Results shown represent mean±SEM. (F) mRNA levels of CSF1 measured by RT-qPCR in MDA-MB-231 cells transduced with CRISPR/Cas9 lentivectors coding for control gRNA or two gRNA against CSF1 (n=3, one representative experiment is shown). (G) CSF-1 levels in CCM or pooled EV-R fractions from MDA-MB-231 deleted for CSF1 as indicated. Quantification done on independent EV isolations is shown. (H) Number of live CD163+CD206+ mo-macs obtained upon 5-day culture of purified CD14+ monocytes from healthy donors with EV-R fractions from equal number of secreting control or CSF1-deleted MDA-MB-231 cells. Each individual donor is shown (n=7). (I) CSF-1 levels in CCM or pooled EV-R fractions from MDA-MB-231, BT-549 and MCF-7 cells. Quantification done on independent EV isolations is shown. (J) CD206+CD163+ positive cells recovered after 5 days or culture of equal amounts of proteins from pooled EV-R fractions from MDA-MB-231, BT-549 or MCF-7 cells with freshly isolated CD14+ monocytes from healthy donors. (K) CSF-1 levels in CCM or pooled EV-R fractions from MCF-7 wild-type cells or MCF-7 overexpressing the full-length CSF-1 protein. Quantification done on independent EV isolations is shown. (L) EVs from control MCF-7 cells or MCF-7 expressing full-length CSF-1 were incubated with CD14+ cells for 5 days and the number of CD206+CD163+ cells at the end of the culture is indicated. Comparison between groups was performed by two-tailed, Wilcoxon test. P values≤0.05 were considered significant and are indicated for each comparison. Each individual donor is shown. Results shown represent mean±SEM.

FIG. 4. Mo-macs induced upon MDA-MB-231 EVs treatment express IFN response genes and are enriched in M1 signature

(A) Number of live CD206+CD163+ cells obtained after 5 days of culture of CD14+ monocytes with equal amounts of CSF-1 on EV-R, EV-P or CCM (0.02 ng/ml) or with rCSF-1 (100 ng/ml) (n=5). (B) Transcriptomic analysis of mo-macrophages differentiated as indicated in (A). Principal component analysis on the 5,000 most variant genes. (C) K-means clustering of differentially expressed genes. (D) Gene Ontology analysis of biological processes enriched in the clusters specific for each type of macrophage (Cluster 5 for EV-R-mo-macs, Cluster 4 for EV-P-mo-macs, Cluster 6 for CSF-1-mo-macs and Cluster 7 for CCM-mo-macs). (E) Scatter Plot of normalized mean expression of M1 and M2 signatures per group.

FIG. 5. Role of STING in recipient monocytes and cGAS in EV-producing tumor cells in IFN response in EV-R-mo-macs

(A) Heat map of ISGs present in cluster 5 from RNAseq K-means clustering analysis (EV-R-mo-macs specific cluster) identified as IFN-related in the GO biological processes analysis. (B) Quantification of CXCL9 and CXCL10 present at day 5 in the supernatant of monocytes treated with rCSF-1, EV-R, EV-P or CCM was evaluated by cytometric bead array (CBA). (C) Expression of IRF7 (left) and percentage of IRF7 positive cells (right) in monocytes treated for 5 days with rCSF-1, EV-R, EV-P or CCM, measured by intracellular staining. (D) Quantification of CXCL9 (left) and CXCL10 (right) present at day 5 in the supernatant of monocytes treated with EV-R+/− STING inhibitor was evaluated by CBA. (E) Percentage of IRF7 positive CD163+CD206+ cells at day 5 in culture of monocytes treated with EV-R or rCSF1+/− STING inhibitor. (F) WB for cGAS and EV-associated proteins done on cell lysates (CL), EV-R and EV-P fractions of MDA-MB-231-SCR-gRNA (Ctrl) or MDA-MB-231-cGAS-gRNA (cGAS). (G) Measurement of cGAMP levels in EV-R and EV-P SEC fractions of MDA-MB-231 control cells or cGAS-deleted cells. (H) EV-R from an equal amount of MDA-MB-231 control (CTRL gRNA) or cGAS-deleted (cGAS gRNA) cells were incubated with CD14+ monocytes for 5 days. At the end of the culture the number of CD163+CD206+ cells was evaluated by FACS (left) and secretion of CXCL10 (middle) and IL-8 (right) was measured by CBA. (I) Percentage of IRF7 and PDL1 positive cells among CD163+CD206+ at the end of the culture of monocytes treated as in (H) with EVs from control cells or cGAS-deleted cells. For (D), (E), (H) and (I), comparison between groups was performed by two-tailed, Wilcoxon test. P values≤0.05 were considered significant and are indicated for each comparison. For (A) and (B) Friedman test for comparison among groups was performed.

FIG. 6. TNBC human tumors release EVs containing CSF-1 and their infiltration with macs containing an EV-R-mo-macs signature confers them better survival probability.

(A) Scheme of tissue-explant culture method for EV isolation from paired tumor tissue and juxta-tumor tissue. EVs were isolated from small volumes of pre-filtered CM by ultracentrifugation. (B) Absolute CSF-1 pg present in 400 μl of conditioned medium as described in (A) or in EVs obtained from 400 μl of conditioned medium. (C) Expression of EV-R-mo-macs- or EV-P-mo-macs-enriched genes in TNBC tumor-infiltrating HLA-DR CD11c″ cells as determined by single cell RNA-seq. UMAP embedding of single cells as per the original study are shown, with colour intensity representing normalized signature expression level. In the right panel, UMAP map of macrophage and monocytes clusters from the HLA-DR CD11c cells scRNAseq analysis from all seven TNBC patients with the identified clusters is shown. Each dot represents a cell, colored by clusters. EV-R and EV-P gene signatures (D) EV-R-mo-macs and EV-P-mo-macs signature expression across breast cancer subsets on the METABRIC cohort (Luminal, n=1314; Her2, n=243; TNBC, n=330). (E) Correlation of the EV-R-signature and the EV-P-signature with established signatures for CD8 cytotoxic, CD8 memory, CD8 exhausted and CD4 T regulatory cells and NK cells in the METABRIC cohort. (F) Assay for migration of total T cells using xCELLigence. T cells were seeded in the upper chamber, and supernatant from rCSF-1-mo-macs or EV-R-mo-macs or EV-P-mo-macs or rCXCL10 in the lower chamber of CIM-plates. Migration was evaluated for 24 hours. (G) Kaplan-Meier curves showing overall survival of TNBC patients from the METABRIC cohort stratified by high (red) and low (blue) expressions of EV-R-mo-macs (left) or EV-P-mo-macs (middle) or of a canonical IFN signature (right). Survival curves were compared with the log-rank test (n=330).

FIG. 7. In vivo experiments on Mice having tumor treated with CSF1-associated EVs as compared to control.

(A) Experimental set up. (B) Tumor volume measured by caliper from d8 after tumor injection in the mammary fat pad, in individual mice. Left panel: control group of mice receiving intratumoral PBS injection. Right panel: experimental group of mice receiving 1.5×109 CSF1-EVs from the MDA-MB-231 human triple-negative breast cancer cells. (C) Luminescence signal in the mammary fat pad area of live mice at d31 after tumor injection, measured using an IVIS Lumina III (Perkin Elmer) imaging system. Left panel: quantification of luminescence signal in each individual mouse; right panel: representative images of 3 mice for each group.

### DETAILED DESCRIPTION OF THE INVENTION

Tumor-associated macrophages (TAMs), which differentiate from circulating monocytes, are pervasive across human cancers and comprise heterogeneous populations. The contribution of tumor-derived signals to TAM heterogeneity is not well understood. In particular, tumors release both soluble factors and extracellular vesicles (EVs), whose respective impact on TAM precursors may be different.

The inventors investigate the role and specific contribution of extracellular vesicles (EVs) in cancer environment. In the present invention, the inventors demonstrate that triple negative breast cancer cells (TNBC) release EVs and soluble molecules promoting monocyte differentiation towards distinct macrophage fates. EVs specifically promoted pro-inflammatory macrophages, bearing a type-I IFN (IFN-I) response signature. The combination of CSF-1 and cGAS/STING ligands carried by TNBC EVs led to differentiation of this particular macrophage subset. Notably, macrophages imprinted with an EV signature were found among patients' TAMs. Furthermore, EV-induced macrophage signature was associated with T cell infiltration and extended patient survival. The inventors uncover novel extracellular vesicles and mechanisms by which tumor cells impact on tumor-associated macrophages in human triple-negative breast cancer. The inventors demonstrate that via specific extracellular vesicles, these tumors promote pro-inflammatory macrophages correlated with better clinical outcome and a better prognosis in TNBC patients. In the present invention, the inventors provide in vitro evidences towards a direct role of EVs as tools, alone or with other immuno-therapies, to promote anti-tumor immune responses. Altogether, the present invention highlights the role of this specific CSF1-associated EV, its use or targeting in the treatment of cancer, inflammatory diseases or autoimmune diseases.

Accordingly, the invention relates to CSF1-associated EV, its use in the treatment and diagnosis of cancer, and its targeting in the treatment of inflammatory diseases and autoimmune diseases.

### CSF1-Associated EV and CSF1-EV-Induced Macrophage

In a first aspect, the invention relates to a CSF1-associated extracellular vesicle (EV), which can be isolated and/or modified, or combined with another compound like but not limited to a therapeutic compound.

Thus, in some embodiments, the invention relates to an isolated or modified CSF1-associated extracellular vesicle (EV).

In some embodiments, the invention relates to a composition, in particular a pharmaceutical composition, comprising the CSF1-associated extracellular vesicle (EV, which can be isolated and/or modified and/or combined.

In another aspect, the present invention relates to CSF1-associated extracellular vesicle (EV) comprising or expressing on its surface an antigen-recognizing receptor or an antigen-recognizing domain such as an antigen-recognizing receptor or an antigen-recognizing domain that specifically or preferentially binds to a tumor-associated antigen or a TAM-associated antigen.

In some embodiments, the invention relates to a CSF1-associated extracellular vesicle (EV) comprising an antigen-recognizing receptor.

In some embodiments, the CSF1-associated extracellular vesicle (EV) comprises an antigen-recognizing receptor or an antigen-recognizing domain that specifically or preferentially binds to a tumor-associated antigen.

In some embodiments, the CSF1-associated extracellular vesicle (EV) comprises an antigen-recognizing receptor or an antigen-recognizing domain that specifically or preferentially binds to a TAM-associated antigen.

In some embodiments, the invention relates to a composition comprising the CSF1-associated extracellular vesicle (EV) comprising on its surface an antigen-recognizing receptor.

In some embodiments, the invention relates to a composition comprising the CSF1-associated extracellular vesicle (EV) comprising on its surface an antigen-recognizing receptor or an antigen-recognizing domain that specifically or preferentially binds to a tumor-associated antigen or a TAM-associated antigen.

The CSF1-associated extracellular vesicle (EV) and the composition of the present invention are each suitable in in vitro uses or ex vivo uses, for research and experimental applications, and in vivo uses, for therapeutic applications and in adoptive cell immunotherapy.

The term “CSF1” has its general meaning in the art and refers to a Colony Stimulating Factor 1, more particularly to human Colony Stimulating Factor 1, a cytokine that controls the production, differentiation, and function of macrophages. CSF1 may correspond to the protein referenced as the Uniprot reference No. P09603. CSF1 is a cytokine that plays an essential role in the regulation of survival, proliferation and differentiation of hematopoietic precursor cells, especially mononuclear phagocytes, such as macrophages and monocytes. CSF1 promotes the release of proinflammatory chemokines.

The term “Extracellular vesicle” or “EV” has its general meaning in the art and refers to complex vehicles of intercellular communication, such as but not limited to exosomes, ectosomes, microvesicles, oncosomes and membrane-enclosed structures or particles such as virus-like particles (VLP) (Chow et al., 2014; Haderk et al., 2017; Wu et al., 2016; Ying et al., 2016). The term “Extracellular vesicle” or “EV” also refers to membrane-enclosed structures that contain proteins and nucleic acids, and can be released into the extracellular environment by all cell types, including cancer cells (Tkach and Théry, 2016; van Niel et al., 2018). Once released, EVs can interact with recipient cells and modulate their function (Cocozza et al., 2020).

The term “EV-associated CSF1” refers to CSF1-bearing EVs. The term “EV-associated CSF1” refers to an EV characterized by the presence of CSF1 on its surface. The term “EV-associated CSF1” also refers to an EV comprising CSF1. The term “EV-associated CSF1” also refers to CSF1-associated EV isolated and characterized such as described in the examples.

The term “macrophages” has its general meaning in the art and refers to a type of leukocyte of the immune system which are mononuclear phagocytes. Macrophages play a critical role in innate and adaptive immunity, as well as in tissue-homeostasis. Macrophages differentiate from embryonic precursors or from circulating monocytes and remain in different tissues including tumors. Macrophages residing in healthy tissues are named Tissue-resident macrophages (TRM). Macrophages infiltrating tumors are named Tumor-associated macrophages or TAM. Macrophages may be defined by various combination of markers as disclosed in the present invention. The term “macrophage” also relates to a monocyte-derived macrophage (MDM). Monocyte-derived macrophages (MDMs), can be generated for example upon CSF1 (M-CSF) or GM-CSF treatment of monocytes.

In another aspect, the present invention relates to the pro-inflammatory macrophages which are activated by the CSF1-associated EV. Said pro-inflammatory macrophages are also named in the present invention CSF1-EV-induced macrophages.

Accordingly, the invention also relates to an isolated or modified macrophage, tumor-associated macrophages (TAM) or a progenitor thereof, wherein said macrophage or progenitor thereof has been co-cultured in vitro with CSF1-associated EV to generate CSF1-EV-induced macrophages.

In another aspect, the invention relates to an isolated or modified macrophage, tumor-associated macrophages (TAM), or a progenitor thereof, encoding an antigen-recognizing receptor, wherein said macrophage, tumor-associated macrophages (TAM) or progenitor thereof has been further co-cultured in vitro with CSF1-associated EV to generate CSF1-EV-induced macrophages encoding an antigen-recognizing receptor.

In one embodiment, the invention relates to an isolated or modified macrophage, tumor-associated macrophages (TAM), or a progenitor thereof, encoding a chimeric antigen receptor (CAR), wherein said macrophage, tumor-associated macrophages (TAM) or progenitor thereof has been further co-cultured in vitro with CSF1-associated EV to generate CSF1-EV-induced CAR-macrophages.

In some embodiments, the invention relates to a composition comprising the isolated or modified macrophage, tumor-associated macrophages (TAM) or progenitor thereof of the invention.

In some embodiments, the invention relates to a composition comprising the CSF1-EV-induced macrophages or the CSF1-EV-induced CAR-macrophages of the invention.

The isolated or modified macrophage, tumor-associated macrophages (TAM) or a progenitor thereof co-cultured in vitro with CSF1-associated EV are also named CSF1-EV-induced macrophage.

The term “CSF1-EV-induced macrophage” refers to activated and immunoresponsive macrophages activated by CSF1-associated EV and associated with T cell infiltration and extended patient survival. The term “CSF1-EV-induced macrophage” also refers to pro-inflammatory macrophages activated by CSF1-associated EV and correlated with better clinical outcome and a better prognosis in cancer patients. The activation of macrophages refers to an induction of a signal transduction or changes in gene expression in the cell resulting in initiation of an immune response. For example, activation of a macrophage may involve activation of an intracellular cascade inducing detectable cell proliferation and/or leading to the initiation of effector functions. CSF1-EV-induced macrophages can thus be associated with T cell infiltration, induced cytokine production, phagocytosis, cell signalling, target cell killing, or antigen processing and presentation. Typically, in response to ligand biding to an antigen-recognizing receptor, a signal transduction cascade is produced. In certain embodiments, when a recombinantly expressed CAR binds to an antigen, a transduction cascade is activated such that an immune response is initiated. The term “CSF1-EV-induced macrophage” also refers to CSF1-EV-induced macrophage isolated, cultured and characterized such as described in the examples.

The term “antigen-recognizing receptor” as used herein refers to a receptor that is capable of activating a macrophage immunoresponsive cell in response to its binding to an antigen such as tumor-associated antigen or TAM-associated antigen. Non-limiting examples of antigen-recognizing receptors include chimeric antigen receptors (“CARs”), and antigen-recognizing receptors that specifically or preferentially bind to a tumor-associated antigen or a TAM-associated antigen (tumor antigen-recognizing receptors or TAM antigen-recognizing receptors).

The terms “tumor-associated antigen” or “TAA” refers to tumor antigen or cancer cell antigen. The terms “tumor-associated antigen” refer to peptides, proteins, glycoproteins or carbohydrates that are specifically or preferentially expressed by cancer cells.

The term “chimeric antigen receptor” or “CAR” as used herein refers to a molecule comprising an extracellular antigen-binding domain that is fused to an intracellular signalling domain that is capable of activating or stimulating a macrophage as herein defined, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signalling domain. In certain embodiments, the CAR is selected to have high binding affinity or avidity for the antigen.

The term “antibody” used herein should be intended in the broadest sense and includes polyclonal and monoclonal antibodies, including full antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., sdAb, sdFv, nanobody) fragments. Unless otherwise stated, the term “antibody” should thus be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD and any origin (such as human camelid or other). In some embodiments the antibody comprises a heavy chain variable region and a light chain variable region. The term “antibody” encompasses whole native antibodies but also recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In certain embodiments, an antibody is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant CL region. The light chain constant region is comprised of one domain, CL. The VH and VL regions can be further sub-divided into regions of hypervariability, termed complementarity determining regions (CDR), interspersed with regions that are more conserved, termed framework regions (FR). Each VH and VL IS composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system.

As used herein, “CDRs” are defined as the complementarity determining region amino acid sequences of an antibody which are the hypervariable regions of immunoglobulin heavy and light chains. See, e.g., Rabat et ak, Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Rabat system (Rabat, E. A., et al. (1991) Sequences of Proteins of Immunological Interest, Fifth Edition, ET.S. Department of Health and Human Services, NIH Publication No. 91-3242).

An “antibody fragment” refers herein to a molecule other than a full antibody that comprises a portion of a full antibody that binds the antigen to which the full antibody binds. Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

As used herein, the term “single-chain variable fragment” or “scFv” is a fusion protein of the variable regions of the heavy (VH) and light chains (VL) of an immunoglobulin covalently linked to form a VH::VL heterodimer. The VH and VL are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See also U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3): 173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

The term “Single-domain antibodies” as used herein are antibody fragments comprising all or a portion of the heavy chain variable domain or all or a portion of the light chain variable domain of an antibody. In certain embodiments, a single-domain antibody is a human single-domain antibody.

The term “antigen” or “Ag” as used herein meant a molecule that provokes an immune response. This immune response may involve either antibody production, or the activation of specific immunoresponsive cells, or both. It must be understood that any macromolecule, including virtually all proteins or peptides, can serve as an antigen. Furthermore, antigens can be derived from recombinant or genomic DNA. Thus, any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. The term “tumor antigen” as used herein refers to any polypeptide expressed by a tumor that is capable of inducing an immune response.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

By “specifically binds” is meant a polypeptide or fragment thereof that recognizes and binds a polypeptide of interest, but which does not substantially recognize and bind other molecules in a sample, for example, a biological sample, which naturally includes a polypeptide of the invention.

As used herein, the terms “peptide”, “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In some embodiments, the cells of the invention express one or more antigen-recognizing receptors on the surface. The cells thus may comprise one or more nucleic acids that encode one or more antigen-specific receptors, optionally operably linked to a heterologous regulatory control sequence. Typically such antigen-specific receptors bind the target antigen with a Kd binding affinity of 10-6 M or less, 10-7 M or less, 10-8 M or less, 10-9 M or less, 10-10 M or less, or 10-11 M or less (lower numbers indicating greater binding affinity).

Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. For example, the nucleic acid encoding the antigen-specific receptor may be heterologous to the immune cell and operatively linked to an endogenous promoter of the T-cell receptor such that its expression is under control of the endogenous promoter.

Among the antigen-specific receptors as per the invention are chimeric antigen receptors (CAR).

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December, 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell. Typically, these receptors are used to graft the specificity of a monoclonal antibody onto an immune cell (e.g. an immunoresponsive cell as defined herein), with transfer of their coding sequence facilitated by viral vectors (typically retroviral vector).

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

The CAR may include:
(a) an extracellular antigen-binding domain,
(b) a transmembrane domain,
(c) optionally a co-stimulatory domain, and
(d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain and a heavy chain variable domain, typically as an scFv.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3(4):388-398 (see notably table 1) and are included in the present application.

Antibodies include chimeric, humanized or human antibodies, and can be further affinity matured and selected as described above. Chimeric or humanized scFv's derived from rodent immunoglobulins (e.g. mice, rat) are commonly used, as they are easily derived from well-characterized monoclonal antibodies. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

Typically, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain variable domain that specifically binds the antigen, such as a cancer marker or cell surface antigen of a cell or disease to be targeted, such as a tumor cell or a cancer cell, such as any of the target antigens described herein or known in the art.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen-specific receptor. Among the antigen-specific receptors are functional non-TCR antigen-specific receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR), the γ subunit of Fc receptor. The transmembrane domain can also be synthetic. In some embodiments, the transmembrane domain is derived from CD28, CD8, CD3-zeta, or the γ subunit of Fc receptor.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3 ζ-chain, which is the primary transmitter of signals from endogenous TCRs. Second generation CARs typically further comprise intracellular signaling domains from various costimulatory protein receptors (e.g., CD28, 41BB (CD28), ICOS) to the cytoplasmic tail of the CAR to provide additional signals to the T cell. Co-stimulatory domains include domains derived from human CD28, 4-1BB (CD137), ICOS-1, CD27, OX 40 (CD137), DAP10, and GITR (AITR). Combinations of two co-stimulatory domains are contemplated, e.g. CD28 and 4-1BB, or CD28 and OX40. Third generation CARs combine multiple signaling domains, such as CD3z-CD28-4-1BB or CD3z-CD28-OX40, to augment potency.

The intracellular signaling domain can be from an intracellular component of the TCR complex, such as a TCR CD3+ chain that mediates T-cell activation and cytotoxicity, e.g., the CD3 zeta chain. Alternative well-suited intracellular signaling domains include the intracellular component of various proteins including but to limited to CD3, the γ subunit of Fc receptor (such as of FcεRIγ), CD64, CD32, CD32b, CD32c, CCD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, or members from the TAM family of phosphatidylserine receptors, such as MerTK (Penberthy, Kristen K, and Kodi S Ravichandran. “Apoptotic cell recognition receptors and scavenger receptors.” Immunological reviews vol. 269,1 (2016): 44-59, but see also Morrissey M A, Williamson A P, Steinbach A M, Roberts E W, Kern N, Headley M B, Vale R D. Chimeric antigen receptors that trigger phagocytosis. Elife. 2018 Jun. 4; 7:e36688), and/or other CD transmembrane domains. The CAR can also further include a portion of one or more additional molecules such as Fc receptor γ, CD8, CD4, CD25, CD16. Typically, TLR signaling domains include the Toll/interleukin receptor homology domain, TIR as well as any intracellular domain interacting with MyDDosome and/or TRIFosome clusters such as in particular with MyD88, TIRAP, TRIF and/or TRAM).

The intracellular signaling domain may also or alternatively comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of the protein. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD35 domain is deleted (residues 90-164). As another example, ITAM1 is substituted with the amino acid sequence of ITAM3, and the remaining CD3 & domain is deleted (residues 90-164). See, for example, Bridgeman et al., Clin. Exp. Immunol. 175(2): 258-67 (2014); Zhao et al., J. Immunol. 183(9): 5563-74 (2009); Maus et al., WO 2018/132506; Sadelain et al., WO/2019/133969, Feucht et al., Nat Med. 25(1):82-88 (2019).

Thus, in some aspects, the antigen binding molecule is linked to one or more cell signaling modules including but not limited to CD3 (in particular CD247, CD3z) and/or modified CD3 (notably modified CD247 or CD3z), the γ subunit of Fc receptor (such as of FcεRIγ), CD64, CD32, CD32b, CD32c, CD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, or members from the TAM family of phosphatidylserine receptors, such as MerTK, and/or other CD transmembrane domains. The CAR can also further include a portion of one or more additional molecules such as Fc receptor Y, CD8, CD4, CD25, CD16. Typically, TLR signaling domains include the Toll/interleukin receptor homology domain, TIR as well as any intracellular domain interacting with MyDDosome and/or TRIFosome clusters such as in particular with MyD88, TIRAP, TRIF and/or TRAM). These one or more signaling domains may be combined with one or more co-stimulatory domains include domains derived, for example, from human CD28, 4-1BB (CD137), ICOS-1, CD27, OX 40 (CD137), DAP10, GITR (AITR), CD80, CD86, CD40, CD16, CD32 and CD64.

In some embodiments, upon ligation of the CAR, the cytoplasmic domain or intracellular signaling domain of the CAR activates at least one of the normal effector functions or responses of the corresponding non-engineered immune cell (typically a phagocytic cell such as a macrophage, a dendritic cell, a monocyte or a granulocyte). For example, the CAR can induce a function of a macrophage, a dendritic cell or a monocyte such as phagocytic activity, cytotoxic activity, or secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen-specific receptor engagement, and/or a variant of such molecules, and/or any synthetic sequence that has the same functional capability.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR adapted for the cells according to the present invention can include one or both of such signaling components.

In some aspects, the CAR includes a primary cytoplasmic signaling sequence that regulates primary activation of the FcR or the Toll-like receptor or any one of CD40, CD64, CD32, CD32b, CD32c, CD16a, CD16bn CD16c, members of the BAI family of phosphatidylserine receptor, such as BAI1, or members from the TAM family of phosphatidylserine receptors, such as MerTK, either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, CD66d, and Jedi-1, and Megf10. In some embodiments, cytoplasmic signaling molecule(s) in the CAR contain(s) a cytoplasmic signaling domain, portion thereof, or sequence derived from CD3 zeta, Jedi-1, or Megf10.

The CAR can also include a signaling domain and/or transmembrane portion of a costimulatory receptor, such as CD28, 4-1BB, OX40, DAP10, and ICOS/CD80, CD86, CD40, CD16, CD32 et and CD64. In some aspects, the same CAR includes both the activating and costimulatory components; alternatively, the activating domain is provided by one CAR whereas the costimulatory component is provided by another CAR recognizing another antigen.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

Among the antigens targeted by the antigen-specific receptors are those expressed in the context of a disease, condition, or cell type to be targeted via the adoptive cell therapy. Among the diseases and conditions are proliferative, neoplastic, and malignant diseases and disorders, more particularly cancers. Infectious diseases and autoimmune diseases, inflammatory diseases or allergic diseases are also contemplated.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1B1 (CYP1B), HER2/neu, p95HER2, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO2011009173 or WO2012135854 and published U.S. application No. US20140065708).

In some aspects, the antigen is expressed on multiple myeloma, such as CD38, CD138, and/or CS-1. Other exemplary multiple myeloma antigens include CD56, TIM-3, CD33, CD123, and/or CD44. Antibodies or antigen-binding fragments directed against such antigens are known and include, for example, those described in U.S. Pat. Nos. 8,153,765; 8,603,477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR.

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11: 152).

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

In some embodiments as provided herein, an immune cell, such as a T cell, can be engineered to repress or disrupt the gene encoding the antigen in the immune cell so that the expressed antigen-specific receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, Ll-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, Ll-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin Al (CCNA1), and/or biotinylated molecules, and/or molecules expressed by HIV, HCV, HBV or other pathogens, such as gp120 (but see also Kuhlmann A S, Peterson C W, Kiem H P. Chimeric antigen receptor T-cell approaches to HIV cure. Curr Opin HIV AIDS. 2018 September; 13(5):446-453).

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cells of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

Example antigen-binding receptors include bispecific antibodies that are macrophage-activating antibodies or T-cell activating antibodies which bind not only the desired antigen but also an activating T-cell antigen such as CD3 epsilon.

In some contexts, the engineered cells include gene segments that cause the cells to be susceptible to negative selection in vivo, such as upon administration in adoptive cell therapy. In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive cell therapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In other embodiments of the invention, the cells of the invention are not engineered to express recombinant antigen-specific receptors, but rather include naturally occurring antigen-specific receptors specific for desired antigens, such macrophages or their progenitors cultured in vitro or ex vivo, e.g., during the incubation step(s), to promote expansion of cells having particular antigen specificity.

### In Vitro or Ex Vivo Method

In another aspect, the present invention also relates to an in vitro or ex vivo method of inducing pro-inflammatory macrophage or generating CSF1-EV-induced macrophage, comprising the step of co-culturing macrophage, tumor-associated macrophages (TAM), or a progenitor thereof with CSF1-associated EV.

In some embodiments, the present invention also relates to an in vitro method of inducing pro-inflammatory macrophage encoding an antigen-recognizing receptor or generating CSF1-EV-induced macrophage encoding an antigen-recognizing receptor, comprising the step of co-culturing macrophage, tumor-associated macrophages (TAM), or a progenitor thereof encoding an antigen-recognizing receptor with CSF1-associated EV.

In some embodiments, the present invention also relates to an in vitro method of inducing pro-inflammatory macrophage encoding a chimeric antigen receptors (CAR) or generating CSF1-EV-induced CAR-macrophage comprising the step of co-culturing macrophage, tumor-associated macrophages (TAM), or a progenitor thereof encoding a CAR with CSF1-associated EV.

In some embodiments, the invention relates to an in vitro method of inducing pro-inflammatory macrophage or generating CSF1-EV-induced macrophage, comprising the steps of:
i) Providing macrophage, tumor-associated macrophages (TAM), or a progenitor thereof,
ii) Providing CSF1-associated EV,
iii) Co-culturing the macrophage, tumor-associated macrophages (TAM), or a progenitor thereof with the CSF1-associated EV.

In some embodiments, the invention relates to an in vitro method of generating CSF1-EV-induced macrophage encoding an antigen-recognizing receptor, comprising the steps of:
iv) Providing macrophage, tumor-associated macrophages (TAM), or a progenitor thereof encoding the antigen-recognizing receptor,
v) Providing CSF1-associated EV,
vi) Co-culturing the macrophage, tumor-associated macrophages (TAM), or a progenitor thereof encoding the antigen-recognizing receptor with the CSF1-associated EV.

In some embodiments, the invention relates to an in vitro method of generating CSF1-EV-induced CAR-macrophage, comprising the steps of:
vii) Providing macrophage, tumor-associated macrophages (TAM), or a progenitor thereof encoding a chimeric antigen receptors (CAR),
viii) Providing CSF1-associated EV,
ix) Co-culturing the macrophage, tumor-associated macrophages (TAM), or a progenitor thereof encoding the CAR with the CSF1-associated EV.

In a further aspect, the present invention also relates to the CSF1-EV-induced macrophage, the CSF1-EV-induced macrophage encoding an antigen-recognizing receptor or the CSF1-induced CAR-macrophage generated by the method of the invention.

In some embodiments, the CSF1-EV-induced macrophage, the CSF1-EV-induced macrophage encoding an antigen-recognizing receptor or the CSF1-induced CAR-macrophage generated by the method of the invention is suitable in both in vitro uses, for research and experimental applications, and in vivo uses, for therapeutic applications and in adoptive cell immunotherapy.

### Therapeutic Method

In a further aspect, the invention relates to CSF1-associated EV for use in the treatment of cancer. In an embodiment, the invention relates to CSF1-associated EV for use in the treatment of breast cancer. In an embodiment, the invention relates to CSF1-associated EV for use in the treatment of Triple Negative Breast Cancer (TNBC).

In some embodiments, the invention relates to CSF1-associated EV comprising an antigen-recognizing receptor for use in the treatment of cancer, in particular breast cancer, more particularly TNBC.

In some embodiments, the invention relates to CSF1-associated EV comprising an antigen-recognizing receptor or an antigen-recognizing domain that specifically or preferentially binds to a tumor-associated antigen for use in the treatment of cancer, in particular breast cancer, more particularly TNBC.

In some embodiments, the invention relates to CSF1-associated EV comprising an antigen-recognizing receptor or an antigen-recognizing domain that specifically or preferentially binds to a TAM-associated antigen for use in the treatment of cancer, in particular breast cancer, more particularly TNBC.

In a further aspect, the invention relates to CSF1-EV-induced macrophage for use in the adoptive cell immunotherapy.

In some embodiments, the invention relates to CSF1-EV-induced macrophage for use in the treatment of cancer, in particular breast cancer, more particularly TNBC.

In a further aspect, the invention relates to CSF1-EV-induced macrophage encoding an antigen-recognizing receptor for use in the adoptive cell immunotherapy.

In some embodiments, the invention relates to CSF1-EV-induced macrophage encoding a chimeric antigen receptor (CAR), herein called CSF1-EV-induced CAR-macrophage, for use in the adoptive cell immunotherapy.

In some embodiments, the invention relates to CSF1-EV-induced macrophage encoding a chimeric antigen receptor (CAR) for use in the treatment of cancer, in particular breast cancer, more particularly TNBC.

The present invention also relates to specific targeting and inhibition of CSF1-associated EV for use in the treatment of inflammatory diseases and autoimmune diseases.

In a further aspect, the invention relates to an inhibitor of CSF1-associated EV for use in the treatment of inflammatory diseases and autoimmune diseases.

In some embodiments, inhibitor of CSF1-associated EV may be performed using bispecific antibodies or antigen binding fragments. Particularly, said bispecific antibodies or antigen binding fragments bind CSF1 and an antigen that is present specifically or preferentially on CSF1-associated EV.

As used herein, the terms “subject”, “individual” or “patient” are interchangeable and refer to a mammal. Typically, a subject according to the invention refers to any subject, preferably human. In some embodiment, the subject is afflicted or at risk to be afflicted with disease associated with immune dysfunction or dysregulation. In some embodiments, the term “subject” refers to a subject afflicted or at risk to be afflicted with cancer. In a particular embodiment, the term “subject” refers to a subject afflicted or at risk to be afflicted with triple negative breast cancer (TNBC). In some embodiments, the term “subject” refers to a subject afflicted or at risk to be afflicted with inflammatory diseases, autoimmune diseases or infectious diseases.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof. In a particular embodiment of the invention, the disease treated or prevented by using a compound according to the invention (e.g. a CSF1-associated EV or CSF1-EV-induced macrophage) is a breast cancer, more particularly a triple-negative breast cancer. In a particular embodiment of the invention, the disease treated or prevented by using a compound according to the invention (e.g. a CSF1-associated EV or CSF1-EV-induced macrophage) is a melanoma. In a particular embodiment of the invention, the disease treated or prevented by using a compound according to the invention (e.g. a CSF1-associated EV or CSF1-EV-induced macrophage) is a fibrosarcoma. In a particular embodiment of the invention, the disease treated or prevented by using a compound according to the invention (e.g. a CSF1-associated EV or CSF1-EV-induced macrophage) is a kung cancer. In a particular embodiment of the invention, the disease treated or prevented by using a compound according to the invention (e.g. a CSF1-associated EV or CSF1-EV-induced macrophage) is a digestive system cancer. In a particular embodiment of the invention, the disease treated or prevented by using a compound according to the invention (e.g. a CSF1-associated EV or CSF1-EV-induced macrophage) is a prostate cancer. In a particular embodiment of the invention, the disease treated or prevented by using a compound according to the invention (e.g. a CSF1-associated EV or CSF1-EV-induced macrophage) is a breast cancer, more particularly an ovarian cancer.

In some other embodiments, the disease associated with immune dysfunction or dysregulation encompass diseases such as autoimmune diseases, inflammatory diseases and infectious diseases.

The term “immune disease” or “autoimmune disease”, as used herein, refers to a condition in a patient characterized by cellular, tissue and/or organ injury caused by an immunologic reaction of the subject to its own cells, tissues and/or organs.

In some embodiments, said disease is chosen from acute or chronic inflammatory, allergic, autoimmune or infectious diseases, graft-versus-host disease, graft-rejection. Non-limiting examples of autoimmune diseases include: type 1 diabetes, arthritis, rheumatoid arthritis, psoriasis and psoriatic arthritis, multiple sclerosis, Systemic lupus erythematosus (SLE or lupus), Inflammatory bowel disease such as Crohn's disease and ulcerative colitis, Addison's disease, Grave's disease, Sjögren's disease, alopecia areata, autoimmune thyroid disease such as Hashimoto's thyroiditis, myasthenia gravis, vasculitis including HCV-related vasculitis and systemic vasculitis, uveitis, myositis, pernicious anemia, celiac disease, Guillain-Barre Syndrome, chronic inflammatory demyelinating polyneuropathy, scleroderma, hemolytic anemia, glomerulonephritis, autoimmune encephalitis, fibromyalgia, aplastic anemia and others. Non-limiting examples of inflammatory and allergic diseases include: neuro-degenerative disorders such as Parkinson disease, chronic infections such as parasitic infection or disease like Trypanosoma cruzi infection, allergy such as asthma, atherosclerosis, chronic nephropathy, diseases or conditions associated with transplant and others. The disease may be allograft rejection including transplant-rejection, graft-versus-host disease (GVHD) and spontaneous abortion.

The term “infectious diseases” has its general meaning in the art and refers to disorders caused by organisms, such as bacteria, viruses, fungi or parasites. The term “infectious disease” also refers to infectious diseases or conditions, such as, but not limited to, viral, retroviral, bacterial, protozoal infections, such as HIV immunodeficiency, Cytomegalovirus (CMV), Epstein-Barr virus (EBV), adenovirus, and BK polyomavirus.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “inhibitor of CSF1-associated EV” refers to any compound selected from the group consisting of but not limited to compounds targeting CSF1 protein and CSF1-associated EV. Inhibitors of CSF1-associated EV are a class of drugs that, preferably reversibly, bind the CSF1 protein and CSF1-associated EV, and thus induce the impairment of CSF-1 signaling and prevent the differentiation of CSF1-EV-induced macrophage, the pro-inflammatory macrophages subset. The term “inhibitor of CSF1-associated EV” refers to compounds that bind to CSF1, particularly CSF1-associated EVs and function as potent inhibitors of the differentiation of monocytes to CSF1-EV-induced macrophage or pro-inflammatory macrophages, the particular macrophage subset induced by the combination of CSF-1 and cGAS/STING ligands carried by EVs, and then induce T cell infiltration. The term “inhibitor of CSF1-associated EV” has its general meaning in the art and refers to a compound that selectively inactivates CSF1 protein, CSF1-associated EV and CSF1 signaling pathway. Typically, an inhibitor of CSF1-associated EV is a small organic molecule, a polypeptide, an aptamer, an oligonucleotide (antisense oligonucleotides, siRNA, shRNA, DNA and RNA aptamers), an antibody, or a bispecific antibody. Compounds targeting CSF1 are well-known in the art as such as described in WO2005046657; WO2002087496 and WO2001030381. Compounds targeting extracellular vesicles are well-known in the art as such as described in Wang et al., 2020; Dong et al., 2018; and Zhang et al., 2018.

The term “inhibitor of CSF1-associated EV” may in particular refer to any compound selected from but not limited to MCS110 antibody, PD-0360324 antibody or derivatives thereof (both are monoclonal antibodies targeting CSF1); AMG820 antibody, Cabiralizumab, IMC-CS4 (LY3022855) antibody, Emactuzumab (RG-7155, RO-5509554) or derivatives thereof (all being monoclonal antibodies targeting CSF1-R), Pexidartinib (CML-261, PLX-3397), ARRY-382, BLZ945, or derivatives thereof (all being small molecules); and compounds described in WO2013119716. In a particular embodiment of the invention, the inhibitor of CSF1-associated EV is a compound that binds to the CSF1-Receptor (CSF1-R), and prevents, inhibits, reduces or impairs a functional interaction between CSF-1 and its receptor CSF-1R. In a particular embodiment of the invention, the inhibitor of CSF1-associated EV is a compound that binds to the CSF1, and prevents, inhibits, reduces or impairs a functional interaction between CSF-1 and its receptor CSF-1R. In a particular embodiment of the invention, the inhibitor of CSF1-associated EV is an antibody, in particular a monoclonal antibody, that binds to the CSF1-Receptor (CSF1-R), and prevents, inhibits, reduces or impairs a functional interaction between CSF-1 and its receptor CSF-1R. In a particular embodiment of the invention, the inhibitor of CSF1-associated EV is an antibody, in particular a monoclonal antibody, that binds to the CSF1, and prevents, inhibits, reduces or impairs a functional interaction between CSF-1 and its receptor CSF-1R.

Tests and assays for determining whether a compound is an inhibitor of CSF1-associated EV (i.e. a compound that prevents, inhibits, reduces or impairs the CSF1 signaling pathway) are well known by the skilled person in the art such as described in Wang et al., 2020 for targeting exosomes in cancer by using competitive binding assays performed for recombinant CSF1 proteins such as fluorescence polarization competitive binding assays and measuring the binding affinities. Determining whether a compound is an inhibitor of CSF1-associated EV may also be performed by measuring the amount of CSF1-EV-induced macrophage, pro-inflammatory macrophages induced by CSF1-associated EVs incubated with monocytes or macrophages with and without said candidate inhibitors, or by measuring soluble factors of macrophages or TAM; by using cancer cell growth inhibitory activity of candidate inhibitors of CSF1-associated EV using luminescent cell viability assays; or by using the methods described in the examples of the present patent application.

In a further aspect, the invention relates to a method of treating cancer comprising the step of administering to the subject a therapeutically effective amount of CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage of the invention.

In a further aspect, the invention relates to a method of treating inflammatory diseases and autoimmune diseases comprising the step of administering to the subject a therapeutically effective amount of an inhibitor of CSF1-associated EV of the invention.

In some embodiments, the CSF1-associated EV, CSF1-associated EV comprising an antigen-recognizing receptor, CSF1-EV-induced macrophage, CSF1-EV-induced macrophage encoding an antigen-recognizing receptor, CSF1-EV-induced CAR-macrophage and/or pharmaceutical composition according to the invention is administered in combination with cancer therapies. In particular, compound and/or pharmaceutical composition of the invention may be administered in combination with targeted therapy, immunotherapy such as immune checkpoint therapy and immune checkpoint inhibitor, co-stimulatory antibodies, chemotherapy and/or radiotherapy.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atezolizumab, durvalumab and avelumab. Example of anti-CTLA-4 antibodies include, but are not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Example of inhibitors of the LAG3 receptor are described in US patent U.S. Pat. No. 5,773,578. Example of KIR inhibitor is IPH4102 targeting KIR3DL2.

In some embodiments, the compound and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor.

Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the compound and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophyllotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound and/or pharmaceutical composition of the invention is administered to the patient in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

### Pharmaceutical Composition

The present invention also relates to a pharmaceutical composition, for example a therapeutic, a vaccine or a veterinary composition, comprising the CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage of the invention. The CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage or the compounds of the invention may be used or prepared in a pharmaceutical composition.

In one embodiment, the invention relates to a pharmaceutical composition comprising the CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage of the invention and a pharmaceutical acceptable carrier for use in the treatment of cancer in a subject of need thereof.

In another embodiment, the invention relates to a pharmaceutical composition comprising the compound of the invention and a pharmaceutical acceptable carrier for use in the treatment of inflammatory diseases and autoimmune diseases in a subject of need thereof.

Typically, said pharmaceutical compositions are formulations for administration, preferably sterile compositions and formulations, such as for adoptive cell therapy. The pharmaceutical composition of the invention generally comprises a sterile pharmaceutically acceptable carrier.

As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can further be incorporated into the compositions. In some aspects, the choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition.

A pharmaceutical composition is formulated to be compatible with its intended route of administration. The pharmaceutical composition can be formulated for any conventional route of administration including a parenteral, intravenous, intramuscular, subcutaneous administration and the like.

Pharmaceutical compositions of the invention may include any further compound which is used in the treatment of cancer.

In one embodiment, said additional active compounds may be contained in the same composition or administrated separately.

In another embodiment, the pharmaceutical composition of the invention relates to combined preparation for simultaneous, separate or sequential use in the treatment of cancer in a subject in need thereof.

In some embodiments, the pharmaceutical composition of the invention relates to combined preparation for simultaneous, separate or sequential use in the treatment of cancer in a subject in need thereof.

The invention also provides kits comprising the compound of the invention. Kits containing the compound of the invention find use in therapeutic methods.

### Diagnostic Method

The present invention shows that the abundance or level of CSF1-associated EV or CSF1-EV-induced macrophage correlates positively with cancer outcome and anti-tumor immunity. Therefore, the level of CSF1-associated EV or CSF1-EV-induced macrophage is a biomarker for the prognosis of cancer useful to predict the outcome of cancer disease in a patient before undergoing cancer treatment or in the course of cancer treatment. Furthermore, antitumor immunity is a predictive factor for cancer treatment efficacy. Therefore, it is considered that the level of CSF1-associated EV or CSF1-EV-induced macrophage is also a biomarker for monitoring cancer treatment useful to predict the response to treatment, in particular a treatment comprising immunotherapy, such as checkpoint blockade therapies, in a cancer patient.

Accordingly, in a further aspect, the present invention relates to a method of prognosis and monitoring of cancer and treatment in a patient, comprising measuring the level of CSF1-associated EV and/or CSF1-EV-induced macrophage in a biological sample from the subject, wherein the level of CSF1-associated EV or CSF1-EV-induced macrophage correlates positively with outcome of cancer disease or treatment in the patient.

In a further aspect, the present invention relates to a method for predicting the outcome of a patient suffering from cancer comprising the steps of:
i) determining the quantity of CSF1-associated EV and/or CSF1-EV-induced macrophage in a biological sample obtained from the patient,
ii) comparing the quantity determined at step i) with their corresponding predetermined reference value, and
iii) detecting differential between the quantity determined at step i) with the predetermined reference value will indicate the outcome of the patient.

CSF1-associated EV may be isolated according to the method disclosed in the present application (see Material and Methods). Macrophage may be isolated according to methods known form the skilled artisan, or according to method disclosed in the present application (see Material and Methods). Quantification of CSF1-associated EV or Macrophages, once isolated, may be performed according to methods known from the skilled artisan, or according to the method disclosed herein. A predetermined reference value may be issued from the results of cohorts of patients whom outcome has been determined by other methods or by observation. The determination of a difference between the quantity of CSF1-associated EV and/or CSF1-EV-induced macrophage in a biological sample obtained from the patient and a predetermined reference may be deduced or determined by comparing the determined value(s) of each measure obtained from said subject with the value(s) associated with the same measure, or the distribution of the value(s) associated with the same measure, in reference subject(s) (for example a healthy subject, or healthy cells of the subject from whom the biological sample is issued, in particular healthy cells issued from the organ of the subject from whom the biological sample is issued), or cohorts of subjects which have already been set up as their likeliness to have a determined outcome, in order to classify the subject into that of those reference cohorts to which it has the highest probability of belonging (i.e. to determining if the subject is likely to heal or not for example).

The term “biological sample” refers to any biological sample derived from the patient such as tumor sample, biopsy sample, cancer sample, lymph node, blood sample, plasma, urine or biofluid.

As used herein, the term “tumor tissue sample” has its general meaning in the art and encompasses pieces or slices of tissue that have been removed including following a surgical tumor resection. The tumor tissue sample can be kept ex-vivo in culture medium for a short-term culture, or subjected to a variety of well-known post-collection preparative and storage techniques (e.g., fixation, storage, freezing, etc.) prior to determining the cell quantities. Typically, the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides).

As used herein, the term “quantity of CSF1-associated EV and/or CSF1-EV-induced macrophage” has its general meaning in the art and refers to the number of CSF1-associated EV and/or CSF1-EV-induced macrophage. The term “quantity of CSF1-associated EV and/or CSF1-EV-induced macrophage” also refers to the density of CSF1-associated EV and/or CSF1-EV-induced macrophage. The term “quantity of CSF1-associated EV and/or CSF1-EV-induced macrophage” also refers to the percentage of CSF1-associated EV and/or CSF1-EV-induced macrophage.

In some embodiment, the present invention relates to a method for predicting the outcome of a patient suffering from cancer comprising the steps of:
i) determining the quantity of CSF1-associated EV and/or CSF1-EV-induced macrophage in a biological sample obtained from the subject,
ii) comparing the quantity determined at step i) with their corresponding predetermined reference value, and
iii) concluding that the patient has a good prognosis when the level determined at step i) is higher than the predetermined reference value or concluding that the patient has a poor prognosis when the level determined at step i) is lower than the predetermined reference value.

As used herein, the term “Good Prognosis” refers to a patient afflicted with cancer that is likely to not present aggressiveness and/or invasiveness of cancer, and/or that is likely to not present recurrence of cancer and/or cancer relapse, and/or that is likely to present a high overall survival (OS), event-free survival (EFS), metastasis-free survival (MFS), and/or Recurrence-free survival (RFS).

As used herein, the term “Poor Prognosis” or “Bad Prognosis” refers to a patient afflicted with cancer that is likely to present aggressiveness and/or invasiveness of cancer, and/or that is likely to present recurrence of cancer and/or cancer relapse, and/or that is likely to present a short overall survival (OS), event-free survival (EFS), metastasis-free survival (MFS), and/or Recurrence-free survival (RFS).

As used herein, the “reference value” refers to a threshold value or a cut-off value. The setting of a single “reference value” thus allows discrimination between a poor and a good prognosis with respect to the aggressiveness, invasiveness and/or recurrence of cancer, cancer relapse and/or overall survival (OS) for a patient. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Particularly, the person skilled in the art may compare the quantity (obtained according to the method of the invention) with a defined threshold value. In one embodiment of the present invention, the threshold value is derived from the quantity (or ratio, or score) determined in a biological sample derived from one or more patients having cancer. Furthermore, retrospective measurement of the quantity (or ratio, or scores) in properly banked historical patient samples may be used in establishing these threshold values.

Predetermined reference values used for comparison may comprise “cut-off” or “threshold” values that may be determined as described herein. Each reference (“cut-off”) value may be predetermined by carrying out a method comprising the steps of
a) providing a collection of samples (such as tumour, blood) from patients suffering of cancer;
b) determining the quantity of CSF1-associated EV and/or CSF1-EV-induced macrophage for each sample contained in the collection provided at step a);
c) ranking the tumour samples according to said quantity of cells;
d) classifying said samples in pairs of subsets of increasing, respectively decreasing, number of members ranked according to their quantity of cells,
e) providing, for each sample provided at step a), information relating to the actual clinical outcome for the corresponding cancer patient (i.e. the duration of the event-free survival (EFS), metastasis-free survival (MFS) or the overall survival (OS) or both);
f) for each pair of subsets of samples, obtaining a Kaplan Meier percentage of survival curve;
g) for each pair of subsets of samples calculating the statistical significance (p value) between both subsets;
h) selecting as reference value for the quantity of cells, the value of the quantity of cells for which the p value is the smallest.

For example, the quantity of CSF1-associated EV and/or CSF1-EV-induced macrophage has been assessed for 100 cancer samples of 100 patients. The 100 samples are ranked according to their quantity of cells. Sample 1 has the best quantity of cells and sample 100 has the worst quantity of cells. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the quantity of cells corresponding to the boundary between both subsets for which the p value is minimum is considered as the reference value. It should be noted that the reference value is not necessarily the median value of quantities of cells.

In routine work, the reference value (cut-off value) may be used in the present method to discriminate cancer samples and therefore the corresponding patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the quantity of cells should of course be used for obtaining the reference value and thereafter for assessment of the quantity of cells of a patient subjected to the method of the invention.

In a particular embodiment, the score may be generated by a computer program.

In one embodiment, the reference value may correspond to the quantity of CSF1-associated EV and/or CSF1-EV-induced macrophage determined in a sample associated with a patient having a good prognosis. Accordingly, a higher or equal quantity of CSF1-associated EV and/or CSF1-EV-induced macrophage than the reference value is indicative of a patient having good prognosis, and a lower quantity of CSF1-associated EV and/or CSF1-EV-induced macrophage than the reference value is indicative of a patient having a poor prognosis.

In another embodiment, the reference value may correspond to the quantity of CSF1-associated EV and/or CSF1-EV-induced macrophage determined in a sample associated with a patient having a poor prognosis. Accordingly, a higher quantity of CSF1-associated EV and/or CSF1-EV-induced macrophage than the reference value is indicative of a patient having good prognosis, and a lower or equal quantity of CSF1-associated EV and/or CSF1-EV-induced macrophage than the reference value is indicative of a patient having poor prognosis.

In some embodiments, the method of the present invention is particularly suitable for predicting the survival time of the patient.

Accordingly, the present invention also relates to a method for predicting the outcome of a patient suffering from cancer comprising the steps of:
i) determining the quantity of CSF1-associated EV and/or CSF1-EV-induced macrophage in a biological sample obtained from the subject,
ii) comparing the quantity determined at step i) with their corresponding predetermined reference value, and
iii) concluding that the patient has a long survival time when the level determined at step i) is higher than the predetermined reference value or concluding that the patient has a short survival time when the level determined at step i) is lower than the predetermined reference value.

The quantity of CSF1-associated EV and/or CSF1-EV-induced macrophage is determined by any well-known method in the art. In some embodiments, the quantity of CSF1-associated EV and/or CSF1-EV-induced macrophage is determined such as described in the example. In some embodiments, the quantity of CSF1-associated EV and/or CSF1-EV-induced macrophage is determined by flow cytometry. In some embodiments, the quantity of CSF1-associated EV and/or CSF1-EV-induced macrophage is determined by IHC or immunofluorescence.

For example, the quantification of the CSF1-associated EV and/or CSF1-EV-induced macrophage is performed by contacting the tumour tissue sample with a binding partner (e.g. an antibody) specific for a cell marker of said cells. Typically, the quantification of the CSF1-associated EV and/or CSF1-EV-induced macrophage is performed by contacting the tissue tumour tissue sample with a binding partner (e.g. an antibody) specific for CSF1-associated EV and/or CSF1-EV-induced macrophage (such as anti-CSF1 antibodies for CSF1-associated EV quantification).

In some embodiments, the quantification of the CSF1-associated EV and/or CSF1-EV-induced macrophage is performed by flow cytometry or Fluorescence-activated cell sorting (FACS). In some embodiments, the quantification of the CSF1-associated EV and/or CSF1-EV-induced macrophage is performed by flow cytometry, Imaging Flow Cytometry or Bead-Based Multiplex Flow Cytometry Assay such as described in the example.

In a further aspect, the method of the present invention is suitable for determining whether a patient is eligible or not to an anti-cancer treatment or an anti-cancer therapy. For example, when it is concluded that the patient has a poor prognosis then the physician can take the choice to administer the patient with an anti-cancer treatment. Typically, the treatment includes chemotherapy, radiotherapy, radioimmunotherapy and immunotherapy.

In a further aspect, the method of the present invention is suitable for determining whether a patient is eligible or not to a treatment with the CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage of the invention. For example, when it is concluded that the patient has a poor prognosis then the physician can take the choice to administer the patient with the treatment of the invention.

In a further aspect, the method of the present invention is suitable for determining whether a patient is responder or not to a cancer therapy or treatment with the CSF1-associated EV, CSF1-associated EV comprising an antigen-recognizing receptor, CSF1-EV-induced macrophage, CSF1-EV-induced macrophage encoding an antigen-recognizing receptor and/or CSF1-EV-induced CAR-macrophage of the invention.

In a further aspect, a method of the present invention is suitable for assessing the likeliness of survivability of a patient having a cancer, said method comprising the steps of:
i) retrieving macrophages, in particular EV-associated Macrophages, more particularly monocyte-derived macrophages (mo-Macs) or EV-mo-Macs in a biological sample previously obtained from a subject having cancer;
ii) measuring the expression of the following genes: SERPING1; GZMA; STAT1; GBP1; RORC; SLC4A10; APOL3; IDO1; ZBED2; SLAMF7; ETV7; GBP4; HAPLN3; GBP5; ZBTB32; CTNND2; ACOD1; CALHM6; ANKRD1; GBP1P1; CXCL9; SIGLEC8; PLA2G2D;
iii) assessing a likeliness of survivability when a majority, in particular at least 12, in particular at least 13, in particular at least 14, in particular at least 15, in particular at least 16, in particular at least 17, in particular at least 18, in particular at least 19, in particular at least 20, in particular at least 21, in particular at least 22, more particularly the 23, genes are over expressed as compared to a control (for example a measure of the expression of the same genes in a healthy subject).

The invention will be further illustrated by the following examples. However, these examples should not be interpreted in any way as limiting the scope of the present invention.

### Example

### Material & Methods

### Cell Culture and Transfections

MDA-MB-231, MCF-7 cells were cultured in Dulbecco's modified Eagle's medium (DMEM-Glutamax, Gibco), with 10% fœtal calf serum (FCS, Gibco), penicillin-Streptomycin (Gibco). BT-549 and THP-1 cells were cultured in RPMI-1640-Glutamax™ medium (Gibco) with Penicillin-Streptomycin and 10% of FCS. CRISPR/Cas9 modified MDA-MB-231 were cultured in complete medium with 2 μg/ml puromycin (ThermoFischer Scientific). MCF-7 cells overexpressing CSF-1 were generated by transduction with recombinant retroviruses (see SI) and kept in culture in complete medium under antibiotic selection (2 μg/ml puromycin).

### CRISPR/Cas9 Edited Cells

STING KO THP1 cells were established by Cas9/gRNA RNP electroporation using predesigned Alt-R CRISPR-Cas9 crRNAs (IDT technologies, see SI). After 24 h at 37° C. cells were expanded. The knockout efficiency was evaluated by western blotting.

cGAS KO, CSF-1 KO or Rab11 KO MDA-MB-231 cells were established by lentiviral-mediated CRISPR/Cas9 method (see SI). Following transduction, cells were selected with puromycin (2 μg/ml). The knockout efficiency was evaluated by RT-qPCR and western blotting and/or LEGENDPlex/ELISA.

### Human Specimens and Processing

Analysis of tumors was performed following relevant national law on protection of people taking part in biomedical research (https://www.sciencedirect.com/topics/medicine-and-dentistry/biomedical-research). Female patients were included in this study and only samples from patients that provided verbal informed consent for tissue donation were processed. Human experimental procedures were approved by the Institutional Review Board and Ethics committee of the Institut Curie Hospital group (approval Feb. 12, 2014) and CNIL (Commission Nationale de l'informatique et des Libertés) (N° approval: 1674356).

Fresh tumor and juxta-tumor tissue were harvested from patients with BC undergoing resection at Institut Curie (Costa et al., 2018). Surgical residues available after pathological analysis and not required for diagnosis were used. For tumor secretome analysis, once resected, the tissue was placed in CO2-independent medium (Gibco) within minutes of collection and submitted for downstream processing and analysis. Tissues were cut into 15 to 20 mg pieces and cultured in one well of a 48-well plate in 250 ml of RPMI GlutaMAX (Gibco) supplemented with 10% FBS (Hyclone), 100 U/ml penicillin/streptomycin (Gibco), 1% MEM Non-Essential Amino Acids (Gibco) and 1% Pyruvate (Gibco) at 37° C. with 5% CO2. After 24 hours, conditioned media were diluted ½ with complete RPMI medium, then filtered with a 0.22 mm filter before storage at −80° C.

### EV Isolation

Cell lines were cultured for 24 h without serum before EVs isolation. The following day, concentrated conditioned medium (CCM) was harvested by pelleting cells at 400×g for 10 min at 4° ° C. Supernatant was centrifuged at 2,000×g for 20 min at 4° C. to discard 2K pellet and then concentrated on a sterilized Sartorius Centrifugal Filter (MWCO=100 kDa; VS2061) or Centricon Plus-70 Centrifugal Filter (MWCO=100 kDa; Millipore). Medium was concentrated to 500 μl and overlaid on 70 nm qEV size-exclusion columns (Izon, SP1) for separation. Twenty two 500 μl fractions were recovered and analyzed separately or pooled, 7 to 10 as EV-Rich (EV-R) fraction and 15 to 22 as EV-Poor (EV-P) fraction. Pooled fractions were then concentrated using 10 KDa cut-off filters (Amicon Ultra-15, Millipore). Protein concentration in EV-R, EV-P or CCM was measured using Micro-BCA (Thermo Scientific) in the presence of 0.2% SDS 200-400 μl of frozen human tumor supernatants from the T-MEGA cohort (see below detailed information on collection) were ultracentrifuged at 100,000×g for 30 min in a TLA-45 rotor (Beckman Coulter). Pellets were re-suspended in 25 μl of PBS.

### EV Characterization

### Western Blot

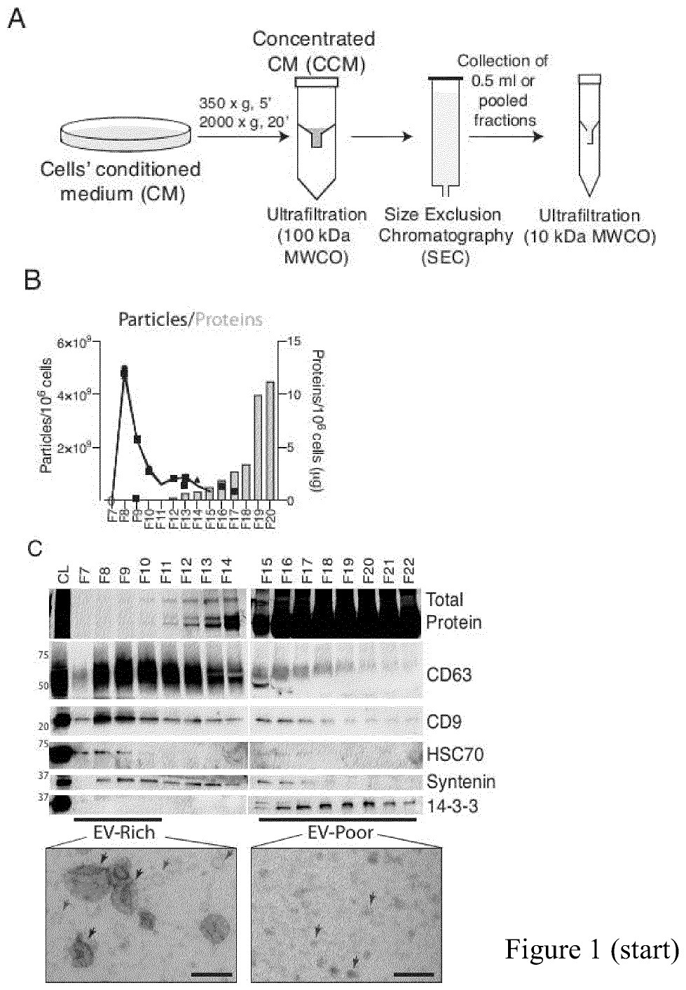

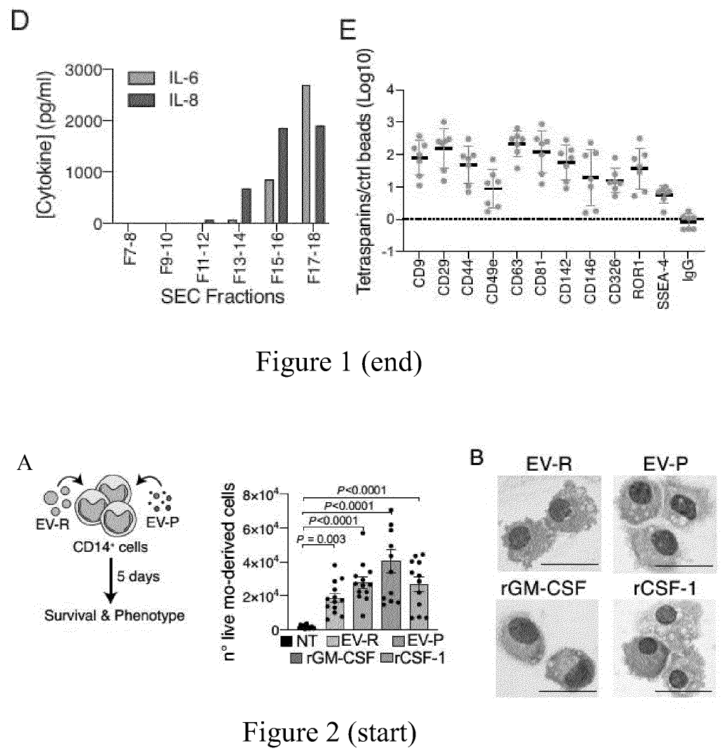

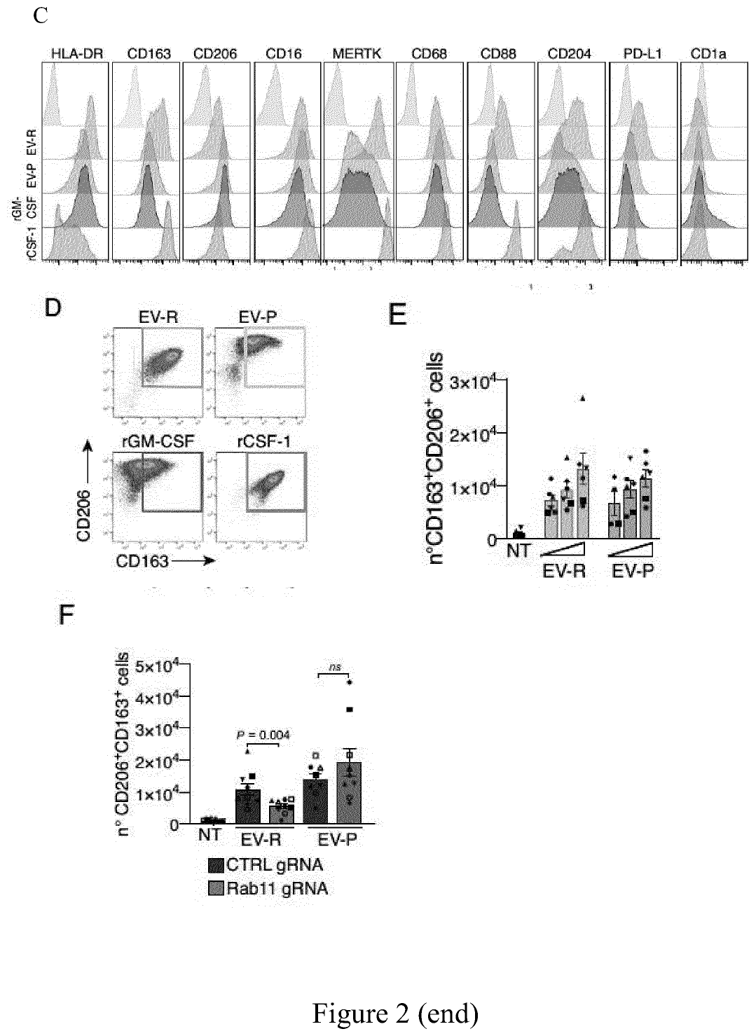

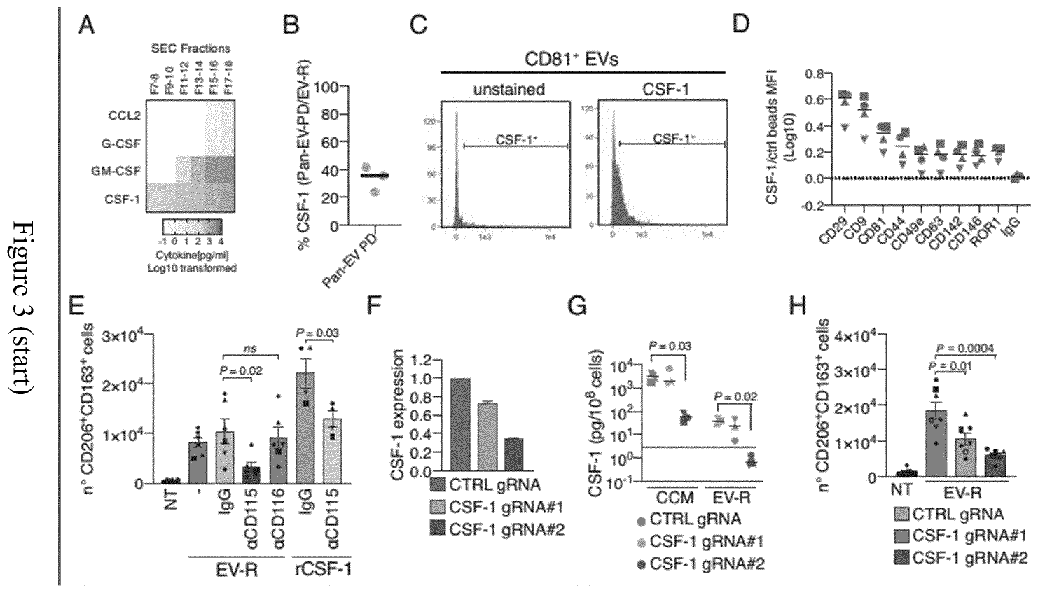

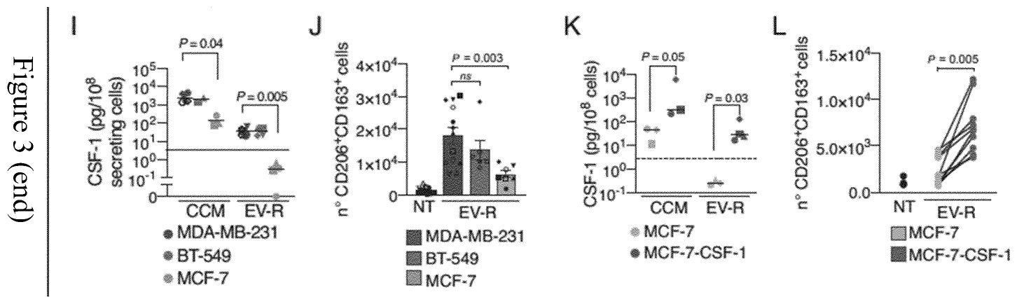

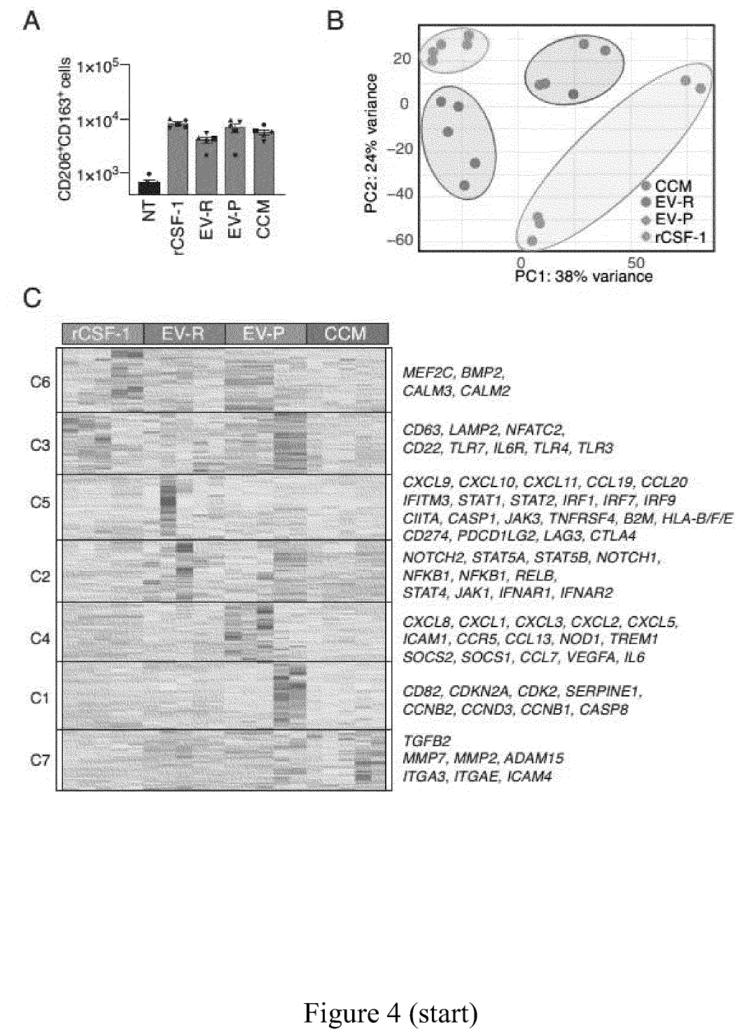

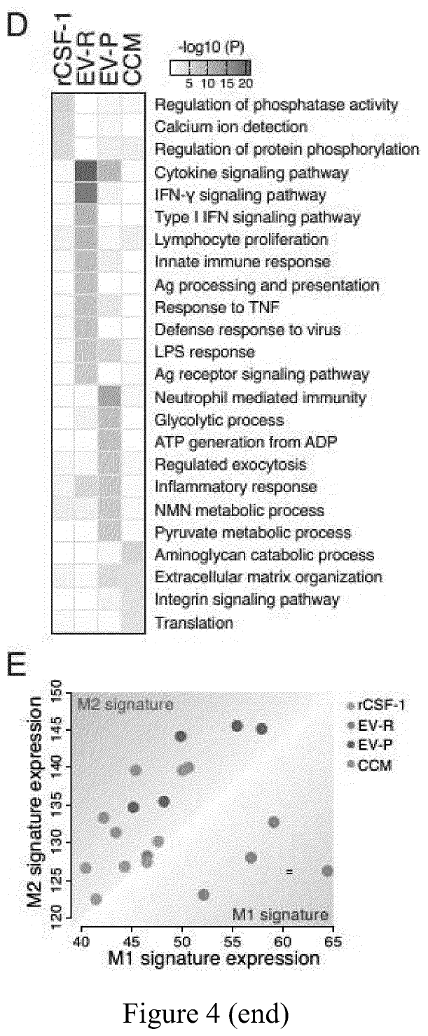

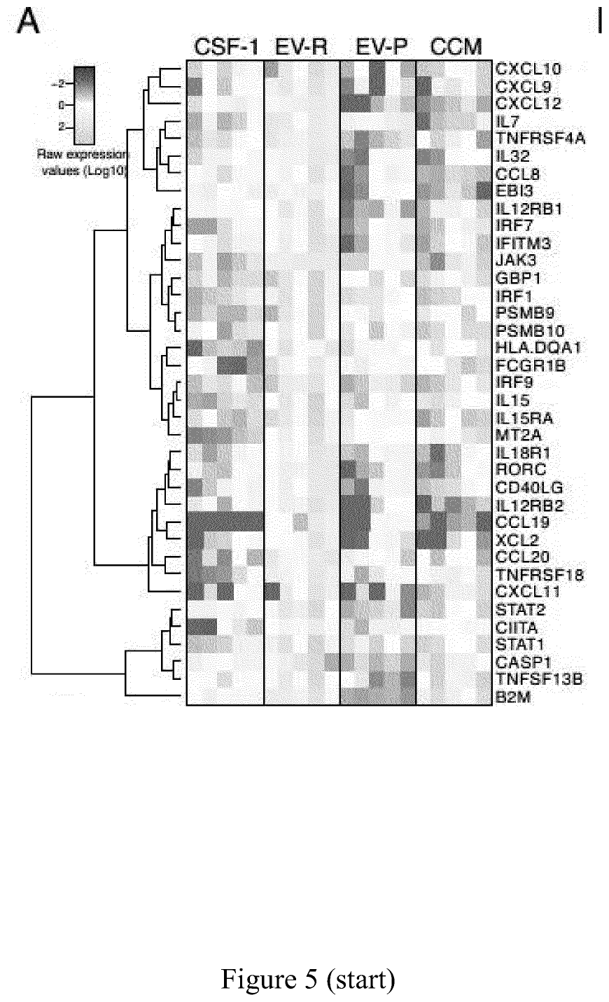

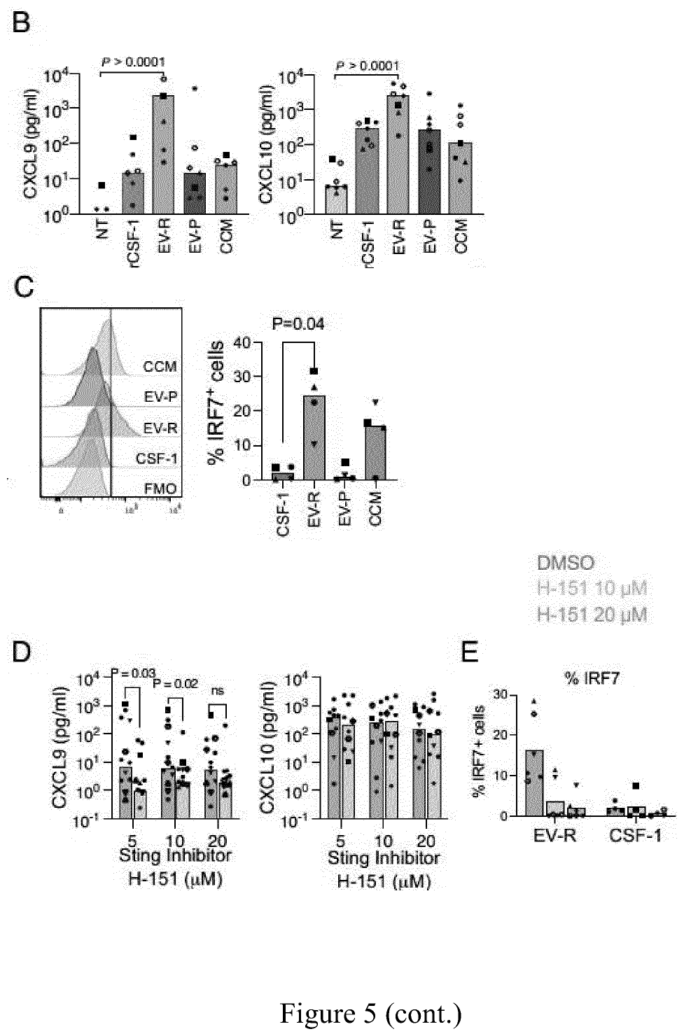

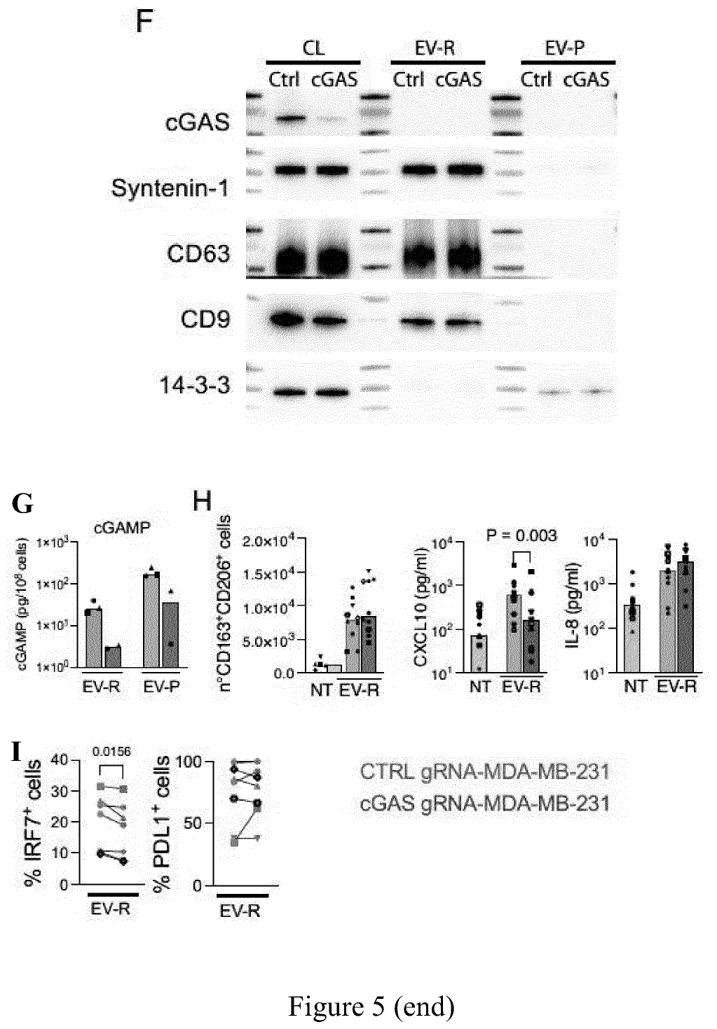

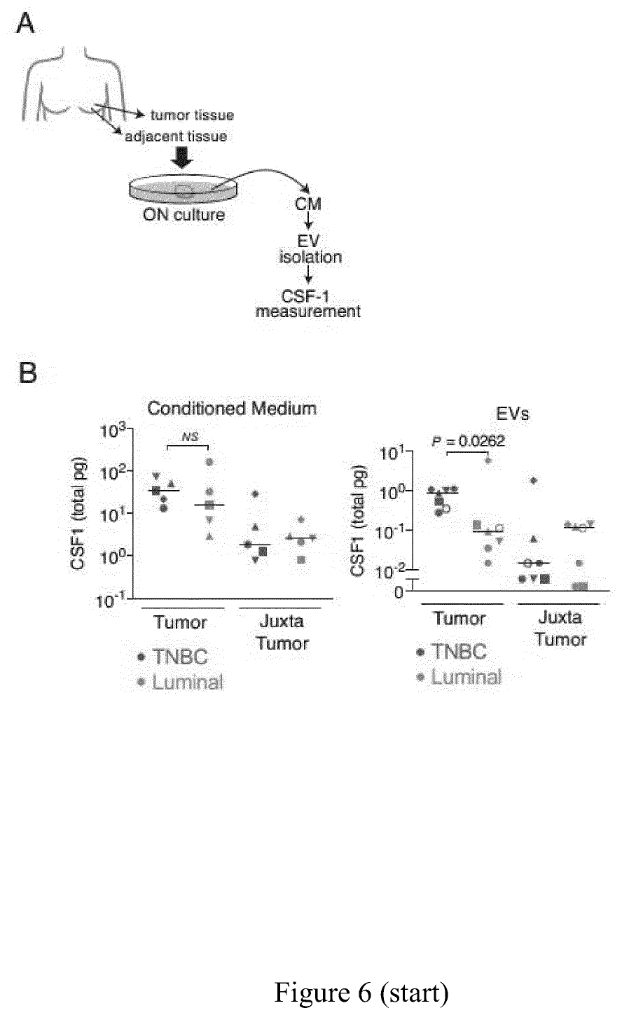

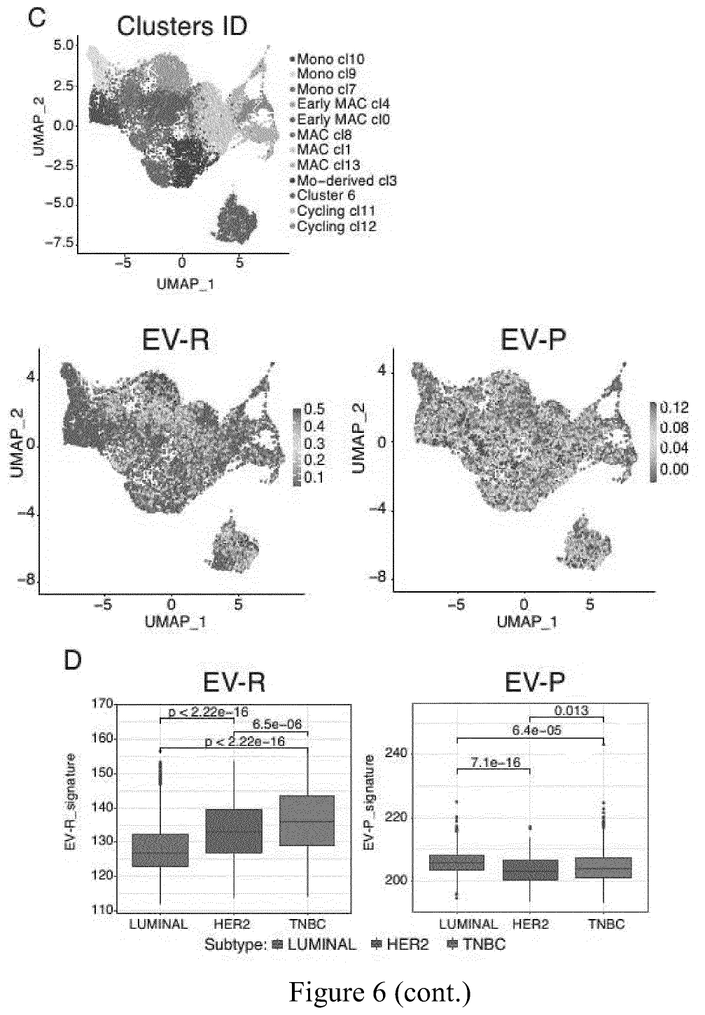

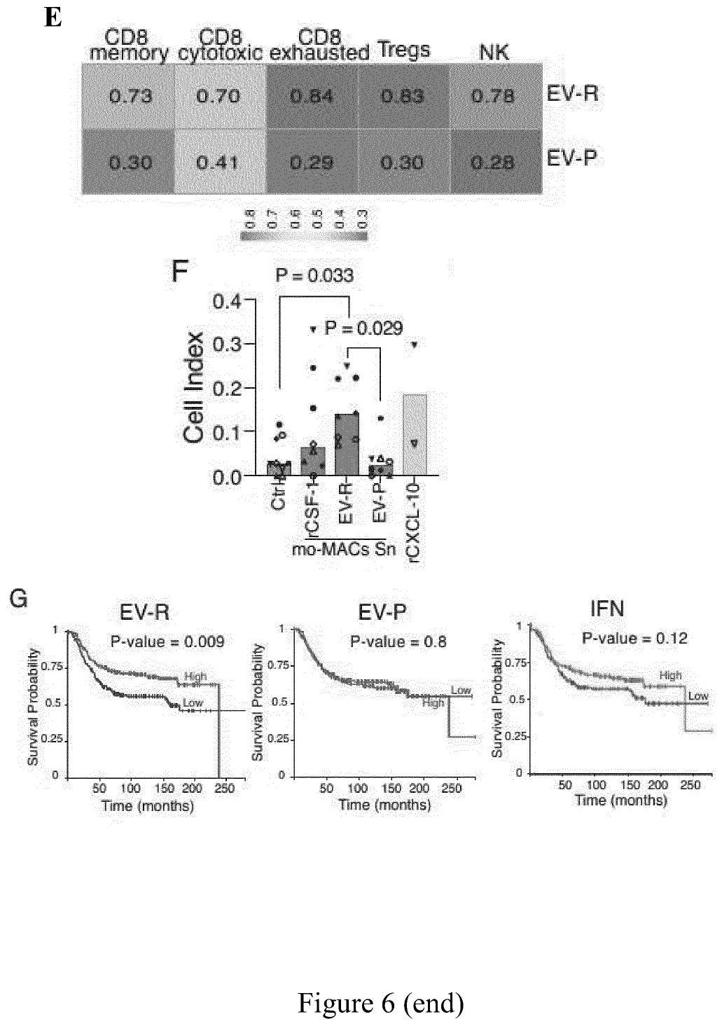

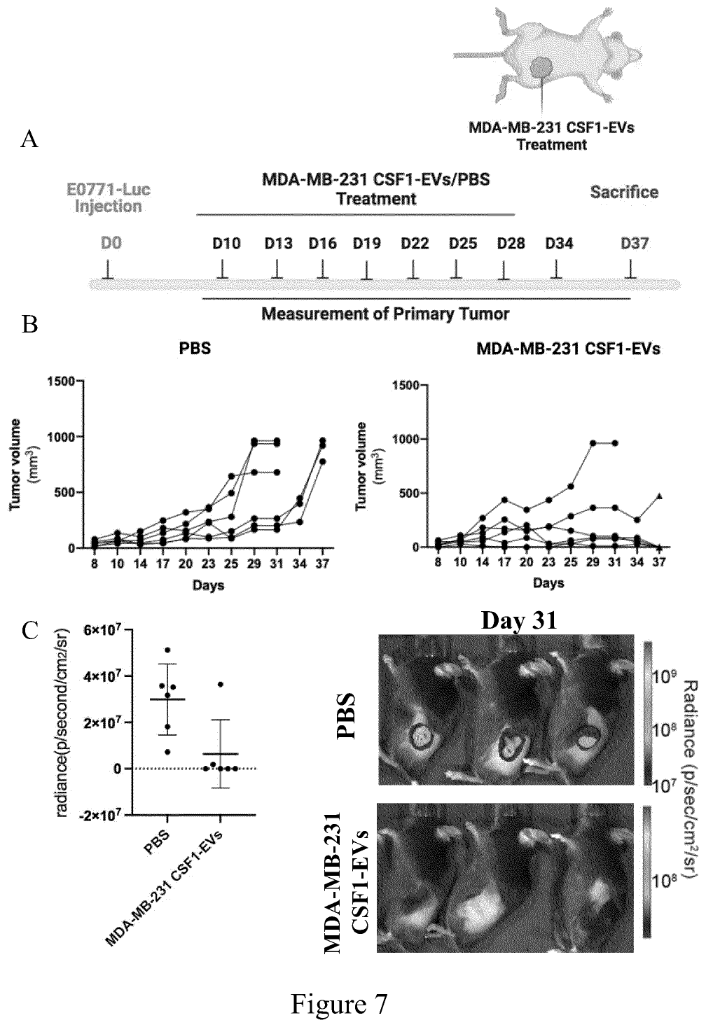

Cell lysates (CL) from 2.105 (FIG. 1C) or 5.104 (FIG. 5F) cells, 20 μL of the 500 μL unconcentrated SEC fractions from 400.106 cells (FIG. 1C) or pooled EV-R and EV-P fractions obtained from 20.106 cells (FIG. 5F) were analysed by Western blot in non-reducing conditions. Membranes were blotted with the following antibodies: mouse anti-human CD63 (clone H5C6, BD Bioscience), mouse anti-human CD9 (clone MM2/57, Millipore), rabbit anti-human 14-3-3 (EPR6380, GeneTex), rat monoclonal anti-human HSC70 (clone 1B5, Enzo Life Sciences), or GP96 (clone 9G10, Enzo Life Sciences). Monoclonal rabbit anti-human syntenin was a gift from P. Zimmermann. Secondary antibodies were purchased from Jackson Immuno-Research

### Bead-Based Multiplex Flow Cytometry Assay

EV-R fractions were subjected to bead-based multiplex analysis by flow cytometry according to manufacturer instructions (MACSPlex Exosome Kit, human, Miltenyi) or anti-CSF1-APC (clone 26786, R&D systems, 1/50) for detection. EV-R fractions from 40 million cells were diluted with MACSPlex buffer to a final volume of 120 μL and 15 μL of MACSPlex Exosome Capture Beads were added. After overnight incubation, washing, and incubation with detection antibodies (APC conjugated anti-CD9, anti-CD63, and anti-CD81), flow cytometric analysis was performed following the acquisition recommendations for the MACSPlex Exosome kit (Miltenyi Biotec). Median fluorescence intensity (MFI) for each capture bead was background corrected by dividing respective MFI values from matched non-EV controls. Values were log 10 transformed. Only normalized log 10 values higher than 0 were considered as positive.

### Immunoisolation

EV-R fraction from 100 million cells was subjected to a tetraspanin (CD9, CD63, CD81) enrichment using Pan Exosome Isolation Kits (Miltenyi Biotech) following manufacturers' instructions. Pulled-down material (PD) from 40 to 80 million cells was used to measure CSF-1 by ELISA. EVs in the Flow-through (FT) were recovered by ultracentrifugation at 100,000×g in a TLA-45 rotor for 20 min (Beckman). PD and FT from 10 million cells were analyzed by WB.

### Imaging Flow Cytometry

10⁸ EVs were analyzed at the single EV level by Imaging Flow cytometry (ImageStream X MKII, Amnis/Luminex) following guidelines and principles described in (Görgen et al.; 2019, with the following labels: lipid dye MemGlow 488 (Cytoskeleton, Inc), anti-CD81-APC (dilution 1/25) (Clone 5A6, Biolegend) and anti-CSF-1-PE (dilution 1/25) (Clone 26786, R&D Systems). Adequate PBS, unstained, Membright488, CD81, CSF-1 monocolors and Fluorescence Minus One (FMOs) EVs controls were performed in parallel.

### Monocyte Isolation and Culture

Blood CD14+ monocytes from healthy donors' PBMC (see SI) (2×105 cells) were cultured for 5 days in complete medium (RPMI-1640-Glutamax™, 10% FCS, 10 mM Hepes (Gibco), 0.1 mM nonessential amino acids (Gibco), 1 mM Sodium Pyruvate (Gibco), 100 U/mL penicillin/streptomycin) with 2-4 μg of proteins or equivalent CSF-1 amounts (0.02 ng/ml) from EV-R or EV-P fractions or CCM or 100 ng/ml of rGM-CSF or 100 ng/ml of rCSF-1 or left untreated. On day 5, supernatants and cells were recovered for analysis. In some experiments, monocytes were pre-treated with increasing amounts of STING-specific inhibitor H-151 (Invivogen) or with antibodies against CSF-1R (Bio Techne, MAB3291-SP) or GM-CSFR (Bio Techne, MAB706-SP) for 1 hour before the addition of the EV-R fraction.

CTRL or STING KO THP1 cells (5×105 cells in 100 μl of RPMI-1640-Glutamax™ with 100 U/mL penicillin, 100 μg/mL streptomycin and 10% FCS) were plated with 0.02 ng/ml of CSF-1 or EV-R fractions overnight at 37° C. or left untreated. Cells and supernatants were recovered for analysis.

### Flow Cytometry

Cells were stained in PBS containing 0.5% BSA and 2 mM EDTA with different combinations of the following primary antibodies: HLA-DR/DQ/DP FITC (Clone REA33, Miltenyi), CD163 PE (clone GHI/61, Biolegend), MerTK PeCy7 (clone 590H11G1E3, Biolegend), CD206 Alexa647 (clone 15-2, Biolegend), PDL1 BV421 (clone 29E.2A3, Biolegend), CD16 PE-Cy7 (clone 3G8, Biolegend), CD14 APC-Cy7 (clone 63D3, Biolegend), CD1a PE-Cy5 (clone HI149, BD), anti-CD88 PE (clone S5/1, Biolegend), CD204 APC (clone 7C9C20, Biolegend), CD68 Pe-Vio770 (clone REA835, Miltenyi) SIGLEC1 APC (REA197, Miltenyi) and IRF7 AF488 (12G9A36, Biolegend) or isotype-matched control antibodies. For IRF7 staining, cells were permeabilized with Transcription Factor Staining Buffer (Thermo). Cells were analyzed on a FACSVerse (BD Biosciences) or MACSQuant (Miltenyi) instrument. Data was analyzed with FlowJo (FlowJo LLC).

### Morphological Analysis

Cells were subjected to cytospin and colored with May-Grunwald/Giemsa staining (Sigma). Pictures were taken with a CFW-1308C color digital camera (Scion Corporation) on a Leica DM 4000 B microscope.

cGAMP Quantification

2′3′-cGAMP ELISA Kit (Cayman Chemical) was used for the quantification of cGAMP in EV-R and EV-P fractions according to the manufacturer's instructions, and as described in (70). After performing the assay, the plate was read at a wavelength of 450 nm. Data was fitted to a 4-parameter sigmoidal curve.

### CSF-1 Gene Expression Quantification

Knock-down efficiency of CSF-1 using CRISPR/Cas9 was evaluated by real-time RT-qPCR using QuantiFast SYBR Green (Qiagen) (see SI). Expression level of mRNA was evaluated by the cycle of quantitation thresholds (Cq) normalized to Cq of GAPDH and the knockdown was calculated as compared with the control cells value.

### RNA-Seq Library Preparation and Analysis

RNA from 5.105-106 cells mo-macs cells was used for sequencing performed using NovaSeq (Illumina) (100-nt-length reads, paired end). Data are accessible through GEO series accession number GSE173771. Differential gene expression analysis was performed using DESeq2 (v1.22.2). Differentially expressed genes between each pair of conditions displaying an adjusted p-value <0.01 and log 2FoldChange >0.5 were kept. The union of these genes was used as input for k-means clustering of gene expression. The gene ontology analysis was performed using Enrichr (https://maayanlab.cloud/Enrichr/). Enrichments were considered statistically significant if they had q-values (i.e., p-values adjusted for multiple testing)<0.05. EV-R-genes involved in cytokine mediated signaling pathway (the most significant GO term) were queried in a database of IFN-regulated genes, Interferome (http://www.interferome.org) Gene signatures for the EV-R-mo-macs and the EV-P-mo-macs groups were generated considering the differentially expressed genes displaying an adjusted p-value<0.01 and log 2FoldChange>2 when compared among all the other RNAseq groups. A canonical IFN-gene signature was generated by curation of the literature (kindly provided by L. Niborski, INSERM U932).

scRNAseq Analysis

Our in-house study of single cell RNASeq on HLA-DR+CD11c+ infiltrating tumors from TNBC patients was used (See Supplementary Info for experimental details. Data deposited in zenodo repository under accession number 5939839). The R package Seurat v3 was used to integrate samples and analyze the datasets. Gene signatures were computed for each cell using AddModuleScore function from Seurat. Briefly this function calculates for each individual cell the average expression of each gene signature, subtracted by the aggregated expression of control gene sets. The calculations were done on the integrated matrix, setting a parameter of 20 control genes.

### Survival Analysis and Correlation Plots

Bulk analyses were performed using METABRIC cohort (Curtis et al., 2012). Statistical tests were performed using unpaired t-tests.

Survival plots were generated using XenaBrowser (https://xenabrowser.net/) using METABRIC cohort. High and low cohorts were split in 2 by the median. Correlation plots were computed using the same METABRIC TNBC cohort, using Spearman correlation between gene sum from the signatures, and plotted using pheatmap R package (v1.0.12).

### Western Blot

Cell lysates (CL) from 0, 2.106 cells or 20 μL of the 500 μL unconcentrated SEC fractions from 400.106 cells were analysed by Western blot in non-reducing conditions (see SI). Membranes were blotted with the following antibodies: mouse anti-human CD63 (clone H5C6, BD Bioscience), mouse anti-human CD9 (clone MM2/57, Millipore), rabbit anti-human 14-3-3 (EPR6380, GeneTex), rat monoclonal anti-human HSC70 (clone 1B5, Enzo Life Sciences), HSC70 and GP96 (clone 9G10, Enzo Life Sciences). Monoclonal rabbit anti-human syntenin was a gift from P. Zimmermann. Secondary antibodies were purchased from Jackson Immuno-Research.

### Migration Assay

Migration assays were performed with the xCELLigence RTCA instrument according to Manufacturer's recommendations. Briefly in the lower chamber of the CIM-16 plate, CM from EV-R-mo-macs, EV-P-mo-macs and CSF-1-mo-macs was added. DMEM 10% FBS, 1% P/S without or with 100 ng/ml of CXCL10 was added as negative and positive control respectively. 40000 CD3 T cells in 100 μl of medium with FBS were added to each well of the upper chamber. Plates were loaded into the xCELLigence RTCA DP instrument inside a 37° C. incubator for 24 hours with readings every 15 minutes. Data was collected and analyzed by RTCA software.

### Cytokines Quantification

CSF-1 and GM-CSF levels in supernatant were measured using the legendplex multiplex assay according to the manufacturer's instructions. Samples were acquired on a BD FACSverse and analyzed using LEGENDplex software (Biolegend). In some experiments, CSF-1 was measured by ELISA (RAB0098 Sigma-Aldrich) following manufacturer instructions.

Cytometric Bead Array (BD CBA Flex Sets) was used for measuring IL-8, IL-6, G-CSF, CCL2 (MCP1), CXCL10 (IP10) and CXCL9 (MIG) in supernatants according to the manufacturer's instructions. Samples were acquired on a BD FACSverse and analyzed with the FCAP Array software.

In Vivo Assay in Mice Injected with E0771 Cells

Female 8-10 weeks old C57B16 mice were injected in the left fourth mammary gland with 5.105 E0771 cells expressing luciferase (E0771-Luc) in 50 μl PBS. When tumors became palpable (Day 10), mice were injected intratumorally with CSF1-EVs isolated from the MDA-MB-231 human triple-negative breast cancer cells (1.5×10⁹ EVs quantified by Nanoparticle Tracking analysis, in 50 μl PBS), or with 50 μl PBS for the Control group. Eight injections were performed 3-4 days apart. Tumor volume was assessed by caliper measurements twice a week for 37 days, using the formula: (width2× length)/2 (mm³). At day 31, tumors were also measured by their luminescence activity quantified in live mice. The Luciferase substrate D-luciferin (Perkin Elmer) was injected intraperitoneally (150 mg/kg), before anesthesia by isoflurane (Zoetis), and bioluminescence imaging 15 min after luciferin injection. The luminescence signal was acquired with an IVIS Lumina III (Perkin Elmer) imaging system and analyzed using the Living Image software (Perkin Elmer). The rate of total light emission of the primary tumor area was calculated and expressed as radiance photons counted during the whole acquisition time.

### Results

Efficient Separation of EVs from Soluble Factors by Size Exclusion Chromatography.

While analyzing EVs from a TNBC cell line interacting with human immune cells, we have observed a major targeting of these EVs to monocytes (Data not shown), suggesting a possible downstream action of EVs on monocyte differentiation. For this experiment, EVs had been obtained by a crude process of differential ultracentrifugation of the cell conditioned medium (CM), known to co-isolate other non-vesicular components (Théry et al., 2018). To minimize the level of soluble proteins co-isolated with EVs, and thus be able to evaluate the specific impact of cancer cell derived EVs on monocyte fate, we implemented a sequential purification protocol (Shu et al., 2020). CM of MDA-MB-231 tumor cells was concentrated by ultrafiltration using 100 kD MWCO filter, followed by size exclusion chromatography (SEC) (FIG. 1A). This enabled us to efficiently separate the components present in the pre-concentrated CM (CCM) (FIGS. 1A and 1B). EVs eluted in fractions 8 to 10, as evidenced by the high concentration of particles measured by Nanoparticle Tracking Analysis (NTA), whereas protein concentration increased from fraction 12 onwards and peaked after fraction 17 (FIG. 1B and total protein gel in FIG. 1C). To characterize the eluted vesicles, we performed Western blot analysis on all the collected SEC fractions and evaluated the presence of classical EV markers. Most particles eluted before F10 (FIG. 1B), and the EV markers CD63 and CD9 were observed mainly in F8-F11 and F8-F9 for CD63 and CD9, respectively; however, we note that both were also detected at lower levels up to F17-19 (FIG. 1C). Similarly, the EV-specific marker HSC70 was only eluted in the first fractions (up to F10), whereas Syntenin-1, which is also associated with EVs, was present from F8 up to F17. Another marker recently attributed to non-exosomal small EVs (Jeppesen et al., 2019), 14-3-3 protein, appeared in the late SEC fractions, thus, not associated with the EV-specific markers (D see FIG. 1C). Further visualization of the pooled EV SEC fractions using transmission electron microscopy (TEM) with negative staining confirmed that early fractions (F7-10) contained a mixture of cup-shaped EVs of 80 to 100 nm, together with smaller round structures of 30-40 nm (see lower part of FIG. 1C). Conversely, late SEC fractions (F15-22) lacked EVs as well as these smaller structures and were enriched in electrodense particles smaller than 25 nm (FIG. 1C). Finally, IL-6 and IL-8, two cytokines known to be secreted by MDA-MB-231, were detected from F13 onward, with higher amounts in F17 and later fractions (FIG. 1D). Collectively, these results demonstrate the suitability of SEC to isolate EV-rich (EV-R: F7-10) and EV-poor (EV-P: F15-22) fractions (FIG. 1C).

We next analyzed the MDA-MB-231-EV-R surface signature using a bead-based assay based on the capture of EVs on antibody-coated beads, which are subsequently detected by flow-cytometry using a combination of three antibodies against CD9, CD81 and CD63 tetraspanins (TSPs) (MACSPlex Exosome (Koliha et al., 2016). Among the 37 surface markers analyzed in this assay, MDA-MB-231 EVs expressed the TSPs CD9, CD63 and CD81, the sialylated glycolipid SSEA-4, several receptors for signaling molecules: ROR1 (receptor for Wnt5a), CD44 (heparan sulfate proteoglycan that can bind cytokines), CD142 (F3, Tissue Factor=initiator of coagulation), and several molecules mediating adhesion to other cells or to extracellular matrix: integrins CD29 (ITGB1), and CD49e (ITGA5), cell adhesion molecules CD146 (MCAM), and CD326 (EPCAM) (FIG. 1E). This complex array of surface molecules could therefore confer EVs a particular way of interacting with their environment, different from the way soluble proteins interact with their targets via a single receptor.

### TNBC-Derived EVs and Soluble Factors Induce Monocyte Differentiation Towards Macrophages

Blood monocytes are plastic cells that can be recruited to tissues during inflammation, giving rise to monocyte-derived (mo-derived) cells, such as TAMs. To evaluate the role of EVs and soluble factors released by tumors in monocyte activation and differentiation, we incubated human blood CD14+ monocytes with MDA-MB-231 tumor cell-derived EV-R or EV-P fractions. In both cases, a higher number of mo-derived cells after five days of culture was obtained, as compared to untreated monocytes (FIG. 2A). Human monocytes do not proliferate in vitro, thus, our results suggest that SEC fractions promote monocyte survival. Notably, the increased survival was comparable to that observed when monocytes were cultured with essential myeloid growth factors (Xue et al., 2014), recombinant macrophage-colony-stimulating factor (M-CSF=CSF-1, gene CSF1) or granulocyte-macrophage CSF (GM-CSF, gene CSF2) (FIG. 2A). Furthermore, phenotypic characterization of these mo-derived cells revealed a typical macrophage morphology for EV-R-, EV-P- and CSF-1-treated monocytes, while this was slightly less evident for GM-CSF-activated monocytes (FIG. 2B). Classical macrophage surface markers, such as CD68 and MERTK (FIG. 2C), were readily detected in all the mo-derived cells. However, we observed differences in their expression levels between mo-derived macrophages (mo-macs) exposed to EV-R (EV-R-mo-macs) or EV-P (EV-P-mo-macs) fractions (FIG. 2C). Notably, EV-R-mo-macs expressed higher levels of CD163, MERTK, CD88, CD204 and PD-L1 and lower levels of the mannose receptor (MRC1/CD206) compared to EV-P-mo-macs (FIG. 2C).

Because TAMs co-express high levels of CD206 and CD163, we decided to focus on the number of live mo-derived cells that expressed these two markers upon EV-R or EV-P in vitro stimulation (FIG. 2D). When compared to macrophages differentiated in vitro with recombinant cytokines, we observed that expression of these markers on EV-R-mo-macs resembled that of CSF-1-induced mo-macs, while EV-P-mo-macs were more similar to the mo-derived cells generated by GM-CSF (FIG. 2D). Finally, the number of CD206+CD163+ mo-macs recovered at the end of the culture was dependent on the amount of EV-R or EV-P (FIG. 2E).

To test directly the role of EVs in macrophage differentiation, we depleted Rab11a by CRISPR/Cas9 as this protein was proposed to be required for the release of EVs (Messenger et al., 2018; Savina et al., 2002). Consistent with observations in other cell types, depletion of Rab11 by CRISPR/Cas9 (Data not shown) decreased the release of EVs, as quantified by total number of particles (Data not shown), and signal for all the EV-specific surface markers detected by the bead-based multiplex assay (Data not shown). Incubation of monocytes with the EV-R fraction derived from Rab11-depleted cells led to a lower number of total recovered cells and of CD206+CD163+ cells (Data not shown) when compared to monocytes exposed to the EV-R fraction obtained from equal number of control cells, whereas the EV-P fractions of WT and Rab11 ko cells led to similar number of CD206+CD163+ cells (FIG. 2F). Collectively, these results demonstrate that both MDA-MB-231-derived EVs and the soluble factors fraction, poor in EVs, can trigger the differentiation of monocytes towards CD206+CD163+ macrophages, although the resulting macrophages display different phenotypes.

### CSF-1 Exposed on TNBC-Derived EVs is Required for Macrophage Differentiation

EVs can carry various cytokines as part of their internal cargo or in association with their surface (Buzás et al., 2018). We thus assessed the presence in SEC fractions of cytokines known to be secreted by tumor cells and to potentially affect monocyte fate. Like IL-6 and IL-8 illustrated in FIG. 1D, G-CSF (CSF3 gene), GM-CSF and CCL2 were absent in EV-R fractions (FIG. 3A). By contrast, CSF-1 was detected both in EV-R and in EV-P fractions. This observation is consistent with the fact that CSF-1 is synthesized as a transmembrane protein and is subsequently cleaved to release the soluble form (Pixley and Stanley, 2004). To determine whether CSF-1 is physically associated with EVs, we isolated EVs by pull-down using beads coated with antibodies against TSPs CD9, CD63 and CD81 (PanEVs) (Data not shown). 30-40% of CSF-1 found in the EV-R is pulled-down together with TSPs-positive EVs (FIG. 3B). To confirm the presence of CSF-1 on the surface of EVs, we analyzed them by imaging flow cytometry (Görgens et al., 2019) (Data not shown) after labelling with Membright-488 (Collot et al., 2019), a fluorescent probe that efficiently stains EV membranes (Hyenne et al., 2019). Using a combination of fluorochrome-conjugated antibodies against CSF-1 and CD81, we observed that ˜40% of CD81+ EVs were positive for CSF-1 (FIG. 3C), while no events positive for both markers were detected in the absence of EVs (Data not shown). To investigate whether other surface markers may be present on CSF-1-positive EVs, we analyzed the EV-R fraction by the multiplex flow cytometry bead-based assay, replacing the anti-TSPs detection antibodies by an antibody against CSF-1 (FIG. 3D). In addition to CD9- and CD81-EVs, we observed that CD29-containing EVs were positive for CSF-1 and, to a lesser extent, CD44-, CD49e-, CD63-, CD142-, CD146- and ROR1-containing EVs as well, when compared to the isotype control (FIG. 3D). Therefore, we conclude that MDA-MB-231-derived EVs transport CSF-1 on their surface.

CSF-1 acts through the cell surface receptor (CD115/CSF-1R), promoting the proliferation, differentiation, and survival of macrophages and their bone marrow progenitors. Given the presence of CSF-1 on the surface of MDA-MB-231 derived EVs and the striking similarity in the CD163/CD206 ratios between EV-R-mo-macs- and rCSF-1-treated cells (FIG. 2C), we reasoned that CSF-1 could be, at least in part, mediating EV-induced differentiation of macrophages. To test this hypothesis, we pre-incubated monocytes with blocking antibodies against CSF-1R (CD115) or GM-CSFR (CD116) before treating them with EV-R fractions. Impairment of CSF-1 signaling dramatically reduced the number of total live cells and of CD206+CD163+ cells recovered at the end of the culture when compared to IgG controls, while, as expected, blocking the receptor for GM-CSF did not affect monocyte survival nor differentiation (FIG. 3E). To directly test the role of tumor CSF-1 in EV-mediated monocyte differentiation, we depleted CSF-1 in MDA-MB-231 cells by CRISPR/Cas9 based on two gRNAs inducing mild (gRNA #1) or strong (gRNA #2) depletion (FIG. 3F). Consistently, the rate of CSF1-deletion correlated well with the levels of CSF-1 secreted into the supernatant and associated to EVs (FIG. 3G). Furthermore, deletion of CSF1 decreased the EV-R-induced mo-macs differentiation (FIG. 3H) and survical. Combined, our results obtained using blocking antibodies and gene-editing-based deletion demonstrate that CSF-1 signaling is required for monocyte differentiation induced by MDA-MB-231-derived EVs.

We then addressed whether EVs from other breast cancer cell lines carried CSF-1 and induced mo-macs differentiation as do MDA-MB-231 EVs. We first looked at the expression of CSF-1 in different breast cancer cell lines within the CCLE database, that we classified into one of the four breast cancer subtypes (luminal A, luminal B, HER2, and basal/TNBC) according to the literature (Dai et al., 2017) (Data not shown). Most of the cell lines with high CSF-1 expression were of basal origin, including MDA-MB-231 cells that are among the highest expressers of CSF-1 mRNA (Data not shown). We chose another TNBC cell line with high level of CSF-1, BT-549, and a luminal A cell line, MCF-7, with low CSF-1 expression. The three cell lines analyzed released a similar amount of EVs (Data not shown). As observed for MDA-MB-231, BT-549 EVs were also positive for CSF-1, while EVs from MCF-7, which released significantly lower amounts of CSF-1 in accordance with the RNAseq data, contained no detectable levels of the protein (FIG. 3I). MCF-7 EVs induced significantly lower numbers of mo-macs when compared to EVs produced by the two TNBC cell lines analyzed (FIG. 3J), similarly to the results obtained upon CSF-1 deletion. By contrast, when we overexpressed CSF-1 on MCF-7 cells, the protein was released in higher amounts in the conditioned medium and was present on EVs (FIG. 3K), which promoted increased mo-macs differentiation (FIG. 3L). Collectively, these results indicate that EVs from cells with high CSF-1 expression, which seems to be a feature of TNBC when compared to luminal cell lines, display CSF-1 molecules on their surface which are necessary for the EV-dependent promotion of mo-macs differentiation.

### EVs Released by TNBC Promote an IFN Response in Macrophages

Considering the findings that CSF-1-containing EV-R and EV-P fractions induced mo-macs with different phenotypes (FIGS. 2C and D), we decided to establish the mo-macs transcriptome profiles, to better understand their similarities and differences. We performed RNAseq on mo-macs differentiated by equal quantities of CSF-1 on EV-R fraction or EV-P fraction (0.02 ng/ml) and compared them to the in vitro mo-macs generated with rCSF-1 (100 ng/ml). In addition, we decided to treat monocytes with the same amounts of CSF-1 in the CCM, to obtain macrophages stimulated both with soluble factors and EVs (FIG. 4A). An amount of recombinant CSF-1 in the range of the concentration used for the endogenous protein failed to differentiate mo-macs (Data not shown), indicating that the recombinant cytokine is 5000 times less efficient than the endogenous one. We analyzed the overall transcriptomic data by identifying genes displaying a significant differential expression between each pair of conditions (adjusted p-value <0.01 and log 2FoldChange >0.5) and by performing a principal component analysis (PCA). Despite some similarities in the level of surface markers among EV-R- and EV-P-mo-macs (Data not shown), they grouped separately in the PCA (FIG. 4B), revealing functionally distinct outcomes on mo-macs differentiation. Using K-means clustering of the differentially expressed genes, we identified seven sets of genes (FIG. 4C). Clusters 7, 6, 5 and 4 were specific for CCM-mo-macs, CSF-1-mo-macs, EV-R-mo-macs and EV-P-mo-macs, respectively. By contrast, clusters 2, 3 and 1 contained genes enriched in cells exposed to two different treatments: EV-R and EV-P for cluster 2, rCSF-1 and EV-R for cluster 3, EV-P and CCM for cluster 1. Gene ontology enrichment analysis (GOEA) revealed that enriched terms in EV-R-mo-macs were associated with cytokine-signaling pathways, in particular to those induced by type II (IFN-γ) and type I Interferon (IFN), lymphocyte activation, and innate immune response. Conversely, EV-P-mo-macs GO-enriched terms related to neutrophil-mediated immunity and metabolic processes (FIG. 4D). Two different activation states are proposed in TAMs: pro-inflammatory M1 macrophages, which are thought to oppose tumor progression, and M2 macrophages that promote tumor growth (Mantovani and Locati, 2013). This polarization model applies to activation states of in vitro generated macrophages; however, its applicability to macrophages found in the tumor microenvironment, where cells were shown to co-express both M1 and M2 associated genes, remains controversial (Azizi et al., 2018). Numerous immunostimulatory genes associated with M1 macrophages (Azizi et al., 2018) were found in the EV-R-mo-macs cluster 5, such as CXCL9, CXCL10 and CXCL11, FCGR1A, IDO1, KYNU, PTPRC (CD45) and LY75 (CD205). However, this cluster also comprised several genes previously associated with M2 macrophages—PDCDILG2 (PD-L2), CD274 (PD-L1) and CCL20. Therefore, we evaluated the expression of the M1 and M2 signatures within the different groups of mo-macs. Consistent with the observed presence of several genes associated with the M1 signature in the EV-R-mo-macs cluster 5, these mo-macs had the highest expression of the M1 signature while they were low in the M2 signature (FIG. 4E). Conversely, EV-P-mo-macs were high in the M2 signature (Azizi et al., 2018), but were likewise high in the M1 signature in some replicates (Data not shown). CCM-mo-macs were slightly higher in M2 signature, and CSF-1-mo-MACS expressed equal low levels of both M1 and M2 signatures.

These findings support the recent idea that macrophage activation in the tumor microenvironment does not necessarily behave as discrete states, since both M1 and M2 associated genes can be induced by tumor-secreted factors. However, we have observed that depending on the nature of the stimuli, EVs or soluble factors or the combination of both, the balance towards one state of activation or the other can be slightly shifted. In addition to the increased PD-L1 and PD-L2 expression in EV-R-mo-macs, other immune checkpoint genes were specifically expressed in these cells, such as CTLA4 and LAG3 (FIG. 4C). Overall, our results demonstrate that tumor cells secrete both EVs and soluble factors that impact differently on monocytes, promoting the generation of distinct macrophage subtypes characterized by their unique transcriptional signatures.

EVs from TNBC Activate IFN Type I Response Genes in Monocytes Partly Through the cGAS/STING Pathway

EVs can carry nucleic acids and proteins that can act as danger-associated molecular patterns (DAMPs) in recipient cells, triggering a rapid activation of signaling pathways that promote inflammation (Robbins and Morelli, 2014). A characteristic response to the detection of DAMPs is the secretion of elevated levels of cytokines, especially IFN type I and the induction of IFN stimulated genes (ISGs). Our RNASeq data indicated the activation of both type I and II IFN pathway in EV-R-mo-macs (FIG. 4D). To study which genes regulated by IFN were induced upon EV treatment, we queried all genes in the cluster 5 specific for EV-R-mo-macs in a database of IFN-regulated genes, Interferome (http://www.interferome.org/) (Rusinova et al., 2013), and analyzed their expression across the different RNAseq samples (FIG. 5A). We observed that the IFN type I response genes coding for the chemokines, CXCL9 and CXCL10, were highly expressed in EV-R-mo-macs at the RNA level. Thus, we validated these findings by measuring at the protein level either the secretion of this anti-viral induced ISGs into the supernatant of the mo-macs culture (FIG. 5B) or the intracellular expression of IRF7 by FACS (FIG. 5C). We observed a stronger induction of CXCL9 and CXCL10 secretion by EV-R than any other treatment (rCSF1, EV-P and CCM), and a specific induction of IRF7 by EV-R and CCM, whereas neither rCSF1 nor EV-P induced its expression.

To better understand what drives the observed IFN response, we evaluated which pathways within monocytes were necessary for the EV-R-induction of ISGs. Activation of the cGAS/STING cytoplasmic DNA sensing pathway results in robust production of type I IFN (Ishikawa et al., 2009; Ishikawa and Barber, 2008). STING is activated upon binding of its ligand, the second messenger 2′3′-cyclic GMP-AMP (cGAMP) which is synthesized by the enzyme cyclic-GMP-AMP synthase (cGAS) in response to cytosolic double stranded (ds)DNA (Wu and Chen, 2014). Since nucleic acids contained within EVs have been proposed to trigger this pathway in recipient cells (Diamond et al., 2018; Kitai et al., 2017; Takahashi et al., 2017), we evaluated whether STING was required for the EV-R-induced ISG response in mo-macs. To assess this, we pre-incubated monocytes with a pharmacological inhibitor of STING (H-151) before the 5-day treatment with MDA-MB-231-derived EVs or rCSF1 and analyzed both macrophage differentiation and cytokine secretion. Although not statistically different, the number of CD206+CD163+ cells recovered at the end of the culture trended lower at the highest dose of STING inhibitor for the EV-R treated cells (Data not shown). This was explained by a reduced expression of CD163 upon inhibitor treatment, while CD206 levels remained unchanged (Data not shown), and the number of live cells at the end of the culture tended to increase in STING-inhibited cells, although not significantly (Data not shown). Contrary to the differences observed on EV-R treated monocytes, CSF-1 induced macrophages differentiation was not affected upon STING inhibition (Data not shown). These observations indicate that STING activation is necessary for CD163 induction by EVs. In addition, cells incubated with the STING inhibitor produced lower levels of CXCL9 upon EV treatment, whereas no significant changes were observed for CXCL10, a chemokine also highly sensitive to NFkB activation, in addition to IFN (Melchjorsen et al., 2003) (FIG. 5D). Upregulation of IRF7 expression was observed upon EV-R (but not upon CSF-1) treatment, and it was abrogated in STING inhibited cells, suggesting that this gene is specifically induced by the STING pathway upon EV-R exposure (FIG. 5E). To further validate these findings, we depleted STING in the monocytic cell line THP-1 by CRISPR/Cas9 (Data not shown). As previously observed for freshly isolated monocytes, chemokine secretion was induced upon treatment of THP-1 cells with MDA-MB-231-EV-R (Data not shown). However, in THP-1, secretion of both CXCL10 and CXCL9 was significantly decreased in STING-depleted cells (Data not shown). Moreover, the IFN-inducible genes SIGLEC1 and IRF7 were highly expressed upon EV treatment of control cells but not of STING-depleted THP-1 cells, which expressed at basal level lower levels of these proteins (Data not shown). In parallel, treatment of THP-1 control cells by rCSF1 did not induce SIGLEC1 or IRF7 expression as did EV-R fractions (Data not shown). Thus, STING in THP-1 recipient cells seems necessary for the EVs to induce the IFN response and secretion of pro-inflammatory chemokines together with a fully differentiated macrophage phenotype, whereas in primary monocytes, STING is required for induction by EVs of some inflammatory genes (e.g. CXCL9 and IRF7), but not all (e.g. CXCL10).

Plausible explanations of this STING-dependent response would be that either dsDNA within EVs is transferred to recipient cell cytosol and directly activates cGAS, or that cGAMP is being transferred within EVs and is directly activating STING in monocytes. Cytosolic cGAMP was shown to spread to bystander cells through gap junctions (Ablasser et al., 2013) or through budding viral particles (Bridgeman et al., 2015; Gentili et al., 2015). Tumor cells, including MDA-MB-231 cells (Carozza et al., 2019), produce and secrete high quantities of cGAMP, which was proposed to be transferred to host non-tumor cells where it triggers STING, resulting in type I IFN production and induction of anti-tumor responses in mouse models (Ahn et al., 2018; Marcus et al., 2018; Schadt et al., 2019). Thus, to explore the possible transport of cGAMP through EVs, we measured cGAMP levels in different SEC fractions of conditioned medium and deleted cGAS in tumor cells by CRISPR/Cas9 (FIG. 5F) to evaluate whether EVs from these cells induced an ISG response in monocytes. We confirmed previous work (Carozza et al., 2019), showing that cGAMP can be released by MDA-MB-231 tumor cells (FIG. 5G). A majority of cGAMP was present in the EV-P fractions, but a detectable portion was found in EV-R fractions with a major decrease upon cGAS deletion in EV-secreting tumor cells (FIG. 5G). EVs were released in overall equal amount (Data not shown) and with similar protein marker profile (FIG. 5F) and EV-associated CSF-1 (Supplementary FIG. 5I) by cGAS-deleted than wild-type MDA-MB-231 cells. EV-R from cGAS-deleted MDA-MB-231 cells had similar abilities to promote survival (Data not shown) and to generate CD206+CD163+ mo-macs when compared to controls (FIG. 5H, left panel). However, these EVs were unable to induce CXCL10 secretion (FIG. 5H, middle panel), while the induction of IL-8, an IFN-independent cytokine, remained unaltered (FIG. 5H, right panel). In addition, EV-R from cGAS-deleted cells induced slightly lower levels of IRF7 when compared to control EVs, while PDL1 levels remained unaltered (FIG. 5I). Similarly, when stimulating THP-1 cells, EVs from cGAS-deleted cells induced lower levels of IRF7 and Siglec-1 (Data not shown) and of CXCL10 secretion (Data not shown) than wild-type EVs. Altogether, these results demonstrate that cGAS-dependent production of cGAMP and its packaging in EVs contribute to the induction of the ISG response triggered by EVs in myeloid cells. Our results, however, do not exclude that, in addition to cGAMP, other cargoes of EVs, including cGAS-independent ones, may also participate in induction of ISG genes, in both STING-dependent and—independent manners

Human TNBC Release CSF-1-Containing EVs and are Infiltrated with Macrophages Enriched in the EV-R-Mo-Macs Signature

To investigate the clinical significance of CSF-1-containing EVs in TNBC and their ability to promote macrophage differentiation, we first evaluated whether EVs containing CSF-1 were released ex-vivo by human tumors. EVs were concentrated from conditioned medium obtained upon overnight culture of resected primary breast tumor or juxta-tumor (non-involved) explants (FIG. 6A). We measured CSF-1 in conditioned medium and observed that similar levels of total secreted CSF-1 were observed for TNBC and luminal tumor samples, while low levels were found for the juxta-tumor tissue (FIG. 6B, left panel). Strikingly, EVs from TNBC tumors contained detectable levels of CSF-1 when compared to luminal tumor-derived EVs, whose measurements were always close to the detection limit of the assay (FIG. 6B, right panel). To assess whether mo-macs cells obtained in our in vitro system resembled cells present in the tumor microenvironment, we selected the most-deregulated genes (Log 2fold change >2) from the EV-R- or EV-P-mo-macs sets of genes (Clusters 5 and 4, respectively, FIG. 4C) to generate an EV-R and an EV-P gene signature (Data not shown), which we used for further comparison with different in vivo RNA expression datasets. First, we analyzed in-house generated single-cell RNAseq (scRNAseq) data of tumor-infiltrating HLA-DR+CD11c+ sorted cells from early-stage treatment-naïve TNBC patients. The scRNAseq dataset was analyzed considering only the monocyte and macrophage clusters (FIG. 6C). Strikingly, the EV-R gene signature was expressed in specific clusters from myeloid-infiltrating TNBC cells in the scRNASeq dataset (FIG. 6C, middle panel). Visualization of the EV-R signature on the scRNAseq UMAP revealed that EV-R-mo-macs mainly resembled a cluster identified as early-macrophages responsive to IFN (Early-MAC-cluster 4) (FIG. 6C, left panel). These results highlight that the EV-R-induced IFN-response is found in an ex vivo dataset, suggesting that TNBC-derived EVs can contribute to shaping the phenotype of cells present in human cancers. Moreover, genes of the EV-P signature were highlighted in the regions of the UMAP representing a cluster of monocytes (Cluster 7) and an early macrophage cluster 0 (FIG. 6C, right panel). Altogether, these results demonstrate that EV-R-mo-macs or EV-P-mo-macs generated in vitro have similar features to different monocyte-derived macrophages subpopulations present in human tumors. We next investigated whether the EV-R-mo-macs and EV-P-mo-macs signatures were associated with clinical outcome in the Molecular Taxonomy of Breast Cancer International Consortium (-METABRIC) cohort (Curtis et al., 2012). We observed a higher expression of the EV-R-mo-macs signature in TNBC when compared to luminal or Her2 subtypes, while the EV-P-mo-macs signature was slightly higher in luminal subset (FIG. 6D). In addition, only the EV-induced signature was correlated with estimated T and NK cell infiltration (Azizi et al., 2018), both for memory, cytotoxic CD8+ T cells and exhausted CD8+ T cells (Guo et al., 2018), and core T regulatory cells (Zemmour et al., 2018) (FIG. 6E). The Chemokines associated to the EV-R-mo-macs, CXCL9 and CXCL10, together with CXCL11 (FIG. 5A), correlate with the level of tumor-infiltrating lymphocytes in human cancers and their secretion by macrophages is required for anti-tumor immune responses following immune checkpoint blockade in breast cancer. Thus, we evaluated the ability of macrophage CM to attract T cells in a migration assay. CM from EV-R-mo-macs was able to induce migration of total T cells when compared to medium alone (FIG. 6F), indicating that chemokines released by mo-macs upon tumor EV treatment promote T cell migration. Finally, when considering the clinical outcome among TNBC patients, the EV-R-mo-macs signature was significantly associated with an improved survival (FIG. 6G, left panel) while EV-P-mo-macs signature had no impact (FIG. 6G, middle panel). The EV-R signature contains only 23 genes of Cluster 5 (FIG. 4C) and the vast majority (21 genes) are not canonical IFN response genes (Data not shown). Notably, TNBC patients expressing high level of the canonical IFN signature were not as strongly associated with improved survival as patients expressing high EV-R-mo-macs signature (FIG. 6G).

Collectively, our results show that TNBCs release CSF-1-exposing EVs that induce monocyte differentiation into a population of macrophages that possess a unique signature associated with a better prognosis.

TABLE 1
List of gene signatures calculated as the differentially
expressed genes with an adjusted p-value <0.01 and
log2FC >2 when compared to all the other RNAseq groups

EV-R
SERPING1; GZMA; STAT1; GBP1; RORC;
SLC4A10; APOL3; IDO1; ZBED2; SLAMF7;
ETV7; GBP4; HAPLN3; GBP5; ZBTB32;
CTNND2; ACOD1; CALHM6; ANKRD1;
GBP1P1; CXCL9; SIGLEC8; PLA2G2D
EV-P
ITGB3; CCL23; CYP3A5; IL17RB; LOC100506178;
CXCL5; CCL7; MMP12; IL6-AS1; IL6; IL7R;
KCNJ15; FAM124A; CA12; SIGLEC12; LINC01132;
PLEKHA7; NDP; MMP1; MAP1LC3C; STEAP1B;
WFDC21P; TREML2; MYOSLID; EGOT; NTSR1; SLC16A10;
DCUN1D3; STRIP2; IL1A; PPBP; NECTIN4;
LINC01537; LIPC-AS1; CH25H; CXCL3;
SLAMF1; CREG2; ENO2; LAMA3; GLI3; KCNN3; NRN1

### Effect on Tumor Growth of CSF1-Associated EVs:

The effect of CSF1-EVs on tumor growth in vivo has been assessed next. Results illustrated on FIG. 7 support the use of CSF1-EVs and CSF1-induced macrophages as an anti-tumor immunotherapeutic tools. Human CSF1 is functional in mouse and allows reconstitution of macrophages in tissues of CSF1-deficient mice (Cecchini et al. 1994). Therefore, the inventors used the CSF1-EVs from MDA-MB-231 triple-negative breast cancer cells, since the inventors had exhaustively characterized them for their ability to induce EV-R-mo-macs with inflammatory signature, in a mouse model of triple-negative mammary carcinoma growing and forming metastases in immunocompetent syngeneic C57B16 mice: E0771. Luciferase-expressing E0771 C57B16 tumor cells were injected at do in the mammary fat pad of C57B16 female mice. Treatments (PBS or CSF1-EVs) were injected every 3-4 days starting at d10. Tumor size was measured by caliper every 3-4 days (FIG. 7B), or by luminescence at d31 (FIG. 7C). E0771 displays an intermediate level of metastasis, expresses mouse CSF1 at low level and is infiltrated by macrophages in vivo (Kim et al. 2019). E0771 cells expressing firefly luciferase were orthotopically grafted in mammary ducts (FIG. 7A). Ten days later, when tumors were palpable, 1.5×10⁹ of CSF1-EVs were injected directly in the tumors, or the same volume of PBS as control. Injections were performed every 3-4 days for 3 weeks. Using caliper measurement of tumor size, it has been observed that CSF1-associated EVs strongly reduced tumor growth (FIG. 7B). By non-invasive luminescence imaging with an IVIS Lumina III device (Perkin Elmer) at day 31 after tumor injection, confirmation that luciferase activity was mostly undetectable in CSF1-EV-injected tumors is illustrated on FIG. 7C. In addition, 2 Mice from each group were sacrificed to quantify the bioluminescence signal of the organs (lung, liver, and spleen) by IVIS imaging ex vivo. It has been observed some luminescence signal, suggesting presence of metastasis in lungs of the 2 mice from the PBS-injected control group, but none in any of these organs in mice treated with CSF-1 EVs. This observation suggests that, in addition to preventing the local growth of tumor, CSF1-associated EVs treatment prevented the metastasis, in the context of a fully functional immune system. These results support the functionality of CSF1-associated EVs as an anti-tumor immunotherapeutic treatment.

### REFERENCES

Throughout this application, various references describe the state of the art to which this invention pertains. The disclosures of these references are hereby incorporated by reference into the present disclosure.
Azizi, E., Carr, A. J., Plitas, G., Cornish, A. E., Konopacki, C., Prabhakaran, S., Nainys, J., Wu, K., Kiseliovas, V., Setty, M., Choi, K., Fromme, R. M., Dao, P., McKenney, P. T., Wasti, R. C., Kadaveru, K., Mazutis, L., Rudensky, A. Y., Pe'er, D., 2018. Single-Cell Map of Diverse Immune Phenotypes in the Breast Tumor Microenvironment. Cell 174, 1293-1308.e36.
Cassetta, L., Fragkogianni, S., Sims, A. H., Swierczak, A., Forrester, L. M., Zhang, H., Soong, D. Y. H., Cotechini, T., Anur, P., Lin, E. Y., Fidanza, A., Lopez-Yrigoyen, M., Millar, M. R., Urman, A., Ai, Z., Spellman, P. T., Hwang, E. S., Dixon, J. M., Wiechmann, L., Coussens, L. M., Smith, H. O., Pollard, J. W., 2019. Human Tumor-Associated Macrophage and Monocyte Transcriptional Landscapes Reveal Cancer-Specific Reprogramming, Biomarkers, and Therapeutic Targets. Cancer Cell 35, 588-602.e10.
Cassetta, L., Pollard, J. W., 2018. Targeting macrophages: therapeutic approaches in cancer. Nat. Rev. Drug Discov. 17, 887-904.
Cecchini, M. G., M. G. Dominguez, S. Mocci, A. Wetterwald, R. Felix, H. Fleisch, O. Chisholm, W. Hofstetter, J. W. Pollard, and E. R. Stanley. 1994. “Role of Colony Stimulating Factor-1 in the Establishment and Regulation of Tissue Macrophages during Postnatal Development of the Mouse.” Development (Cambridge, England) 120 (6): 1357-72.
Chevrier, S., Levine, J. H., Zanotelli, V. R. T., Silina, K., Schulz, D., Bacac, M., Ries, C. H., Ailles, L., Jewett, M. A. S., Moch, H., van den Broek, M., Beisel, C., Stadler, M. B., Gedye, C., Reis, B., Pe'er, D., Bodenmiller, B., 2017. An Immune Atlas of Clear Cell Renal Cell Carcinoma. Cell 169, 736-749.e18.
Chow, A., Zhou, W., Liu, L., Fong, M. Y., Champer, J., Van Haute, D., Chin, A. R., Ren, X., Gugiu, B. G., Meng, Z., Huang, W., Ngo, V., Kortylewski, M., Wang, S. E., 2014. Macrophage immunomodulation by breast cancer-derived exosomes requires Toll-like receptor 2-mediated activation of NF-κB. Sci. Rep. 4, 5750.
Cocozza, F., Grisard, E., Martin-Jaular, L., Mathieu, M., Théry, C., 2020. SnapShot: Extracellular Vesicles. Cell 182, 262-262.el.
Dong H, Wang W, Chen R, Zhang Y, Zou K, Ye M, He X, Zhang F, Han J. Exosome-mediated transfer of lncRNA-SNHG14 promotes trastuzumab chemoresistance in breast cancer. Int J Oncol. 2018 September; 53(3):1013-1026.
Franklin, R. A., Liao, W., Sarkar, A., Kim, M. V, Bivona, M. R., Liu, K., Pamer, E. G., Li, M. O., 2014. The cellular and molecular origin of tumor-associated macrophages. Science 344, 921-925.
Goudot, C., Coillard, A., Villani, A. C., Gueguen, P., Cros, A., Sarkizova, S., Tang-Huau, T. L., Bohec, M., Baulande, S., Hacohen, N., Amigorena, S., Segura, E., 2017. Aryl Hydrocarbon Receptor Controls Monocyte Differentiation into Dendritic Cells versus Macrophages. Immunity 47, 582-596.e6.
Haderk, F., Schulz, R., Iskar, M., Cid, L. L., Worst, T., Willmund, K. V, Schulz, A., Warnken, U., Seiler, J., Benner, A., Nessling, M., Zenz, T., Göbel, M., Dürig, J., Diederichs, S., Paggetti, J., Moussay, E., Stilgenbauer, S., Zapatka, M., Lichter, P., Seiffert, M., 2017. Tumor-derived exosomes modulate PD-L1 expression in monocytes. Sci Immunol 2.
Kim, Ik Sun, Yang Gao, Thomas Welte, Hai Wang, Jun Liu, Mahnaz Janghorban, Kuanwei Sheng, et al. 2019. “Immuno-Subtyping of Breast Cancer Reveals Distinct Myeloid Cell Profiles and Immunotherapy Resistance Mechanisms.” Nature Cell Biology 21 (9): 1113-26. https://doi.org/10.1038/s41556-019-0373-7.
Kugeratski, F. G., Kalluri, R., 2021. Exosomes as mediators of immune regulation and immunotherapy in cancer. FEBS J. 288, 10-35.
Lavin, Y., Kobayashi, S., Leader, A., Amir, E. A. D., Elefant, N., Bigenwald, C., Remark, R., Sweeney, R., Becker, C. D., Levine, J. H., Meinhof, K., Chow, A., Kim-Shulze, S., Wolf, A., Medaglia, C., Li, H., Rytlewski, J. A., Emerson, R. O., Solovyov, A., Greenbaum, B. D., Sanders, C., Vignali, M., Beasley, M. B., Flores, R., Gnjatic, S., Pe'er, D., Rahman, A., Amit, I., Merad, M., 2017. Innate Immune Landscape in Early Lung Adenocarcinoma by Paired Single-Cell Analyses. Cell 169, 750-765.e17.
Qian, B. Z., Li, J., Zhang, H., Kitamura, T., Zhang, J., Campion, L. R., Kaiser, E. A., Snyder, L. A., Pollard, J. W., 2011. CCL2 recruits inflammatory monocytes to facilitate breast-tumour metastasis. Nature 475, 222-225.
Robbins, P. D., Morelli, A. E., 2014. Regulation of Immune Responses by Extracellular Vesicles. Nat Rev Immunol 14, 195-208.
Tkach, M., Kowal, J., Théry, C., 2018. Why the need and how to approach the functional diversity of extracellular vesicles. Philos. Trans. R. Soc. B Biol. Sci. 373, 20160479.
Tkach, M., Théry, C., 2016. Communication by Extracellular Vesicles: Where We Are and Where We Need to Go. Cell 164, 1226-1232.
van Niel, G., D'Angelo, G., Raposo, G., 2018. Shedding light on the cell biology of extracellular vesicles. Nat. Rev. Mol. Cell Biol. 19, 213-228.
Wang L, Wang B, Wen H, Mao J, Ren Y, Yang H. Exosomes: A rising star in breast cancer (Review). Oncol Rep. 2020 August; 44(2):407-423.
Wu, L., Zhang, X., Zhang, B., Shi, H., Yuan, X., Sun, Y., Pan, Z., Qian, H., Xu, W., 2016. Exosomes derived from gastric cancer cells activate NF-κB pathway in macrophages to promote cancer progression. Tumour Biol. 37, 12169-12180.
Ying, X., Wu, Q., Wu, X., Zhu, Q., Wang, Xinjing, Jiang, L., Chen, X., Wang, Xipeng, 2016. Epithelial ovarian cancer-secreted exosomal miR-222-3p induces polarization of tumor-associated macrophages. Oncotarget 7, 43076-43087.
Zhang P, Zhou H, Lu K, Lu Y, Wang Y, Feng T. Exosome-mediated delivery of MALAT1 induces cell proliferation in breast cancer. Onco Targets Ther. 2018 Jan. 9; 11:291-299.

1. An isolated or modified CSF1-associated extracellular vesicle (EV).

2. The CSF1-associated EV of claim 1 comprising an antigen-recognizing receptor.

3. The CSF1-associated EV of claim 2, wherein the antigen-recognizing receptor binds to a tumor-associated antigen or a TAM-associated antigen.

4. An isolated or modified macrophage, tumor-associated macrophages (TAM) or progenitor thereof, wherein said macrophage or progenitor thereof has been co-cultured in vitro with a CSF1-associated EV to generate CSF1-EV-induced macrophages.

5. The CSF1-EV-induced macrophage according to claim 4 which further encodes an antigen-recognizing receptor.

6. The CSF1-EV-induced macrophage of claim 5, wherein the antigen-recognizing receptor is a chimeric antigen receptors (CAR) or binds to a tumor-associated antigen.

7. A method of adoptive cell immunotherapy comprising administering the CSF1-EV-induced macrophage of claim 4 to a human subject.

8. A method of treating cancer comprising administering the CSF1-associated EV of claim 1 to a human subject.

9. The method of claim 4 wherein the cancer is breast cancer.

10. The method of claim 8, wherein the CSF1-associated EV is administered in combination with immunotherapy.

11. A pharmaceutical composition comprising the CSF1-associated EV claim 1 and a pharmaceutically acceptable carrier.

12. (canceled)

13. An in vivo or ex vivo method for predicting the outcome of a patient suffering from cancer comprising the steps of:
(i) determining the quantity of CSF1-associated EV and/or CSF1-EV-induced macrophage in a biological sample obtained from the patient,
(ii) comparing the quantity determined at step (i) with their corresponding predetermined reference value, and
(iii) detecting differential between the quantity determined at step (i) with the predetermined reference value will indicate the outcome of the patient.

14. CSF1-EV-induced macrophages obtained from a macrophage, a tumor-associated macrophages (TAM) or progenitor thereof, co-cultured in vitro with a CSF1-associated EV.

15. A pharmaceutical composition comprising the CSF1-EV-induced macrophage of claim 4 and a pharmaceutically acceptable carrier.
